(19) United States
(12) Patent Application Publication
(10) Pub. No.: US 2011/0105608 A1
(43) Pub. Date: May 5, 2011

(54) MODULATORS OF NUCLEAR RECEPTOR CO-REGULATORY PROTEIN BINDING

(75) Inventor: Robert N. Hanson, Newton, MA (US)

(73) Assignee: NORTHEASTERN UNIVERSITY, Boston, MA (US)

(21) Appl. No.: 12/933,706

(22) PCT Filed: Mar. 23, 2009
(86) PCT No.: PCT/US09/37986

§ 371 Date: Jan. 10, 2011

Related U.S. Application Data
(60) Provisional application No. 61/038,517, filed on Mar. 21, 2008.

(51) Int. Cl. A61K 31/24; C07C 69/616; C12N 5/00; A61P 35/00; C07C 57/46; A61K 31/195

(52) U.S. Cl. 514/539; 560/36; 435/375; 562/441; 514/567

(57) ABSTRACT

Disclosed are novel compounds and compositions for inhibition of androgen and estrogen receptor signaling, methods for inhibiting androgen signaling, methods for inhibiting estrogen signaling, methods for inhibiting the interaction between a co-regulatory protein and an androgen or estrogen receptor, and methods for treating cancer.

### PRIORITY CLAIM

This disclosure claims the benefit of U.S. Provisional Application No. 61/038,517, filed Mar. 21, 2008. The entire disclosure of that application is relied on and incorporated into this application by reference.

### FIELD OF THE INVENTION

The present disclosure relates to the fields of medicinal chemistry and biology, and more particularly to regulation of androgen and estrogen receptors.

### BACKGROUND

The uptake of steroid hormones, such as androgens and estrogens, into the cell, is followed by passage into the nucleus where they bind to the chaperoned receptor causing release of heat shock proteins (Hsp), and dimerization of the receptor. This process initiates two key events. One is the exposure of the zinc fingers in the DNA-binding domain (DBD) responsible for binding to the androgen response element (ARE) or estrogen response element (ERE) on DNA. The second event is the formation of the nuclear receptor box (NRB) binding site to which the co-regulatory proteins bind. Co-regulatory proteins (co-activators and co-repressors) contain an amphipathic α-helical sequence characterized by an LXXLL motif, where L=leucine and X=any amino acid including leucine. The individual proteins vary with respect to their flanking and internal XX residues, thereby providing the cellular and context selectivity necessary to regulate the particular physiological response. As a result, compounds that are selective for one motif may modulate a very limited subset of hormone responses while leaving the others largely unaffected.

Prostate cancer is the most common cancer diagnosis and the second leading cause of death in men. The majority of these cases are hormone (androgen) dependent and defined as having elevated levels of androgen receptors (AR) and requiring circulating androgens to maintain tumor growth. Treatment typically involves a combination of surgery (partial or total prostatectomy) and radiation of the affected tissue and is often followed by endocrine (hormone deprivation) chemotherapy with an anti-androgen, such as casodex, a LHRH-antagonist, such as leuprolide, or a 5α-reductase inhibitor.

Unfortunately, resistance to the chemotherapy develops often in 2-4 years resulting in the need for more powerful, and non-AR-targeted therapy. In addition, use of anti-androgens is often accompanied by side effects that either limit its effectiveness or seriously compromise the quality of life for the patient.

Breast cancer is the most common cancer diagnosis and the second leading cause of death in women. The majority of these cases are hormone (estrogen) dependent, defined as having elevated levels of estrogen receptors (ER) and requiring circulating estrogens to maintain tumor growth. Treatment typically involves surgery (partial or total mastectomy) and radiation of the affected tissue and is often followed by endocrine chemotherapy with an anti-estrogen, such as tamoxifen, raloxifene or faslodex, or an aromatase inhibitor.

Unfortunately, resistance to the chemotherapy develops often in 2-4 years, resulting in the need for more powerful, and non-ER-targeted therapy. In addition, use of anti-estrogens is often accompanied by side effects that either limits its effectiveness or seriously compromise the quality of life for the patient.

Thus, a need exists for new, anti-hormonal agents that can be used in conjunction with, or instead of, current drugs, that are more effective than current treatments, that have fewer side effects, and that would significantly enhance the treatment of the many women who present with hormone-responsive breast cancer and the many men who present with hormone-responsive prostate cancer.

### SUMMARY OF THE INVENTION

It has been discovered that certain proteomimetic agents can selectively and effectively block the interaction between the ligand-bound AR and the cognate AR-coregulatory protein in prostate cancer cells. It has also been discovered that these proteomimetic agents can selectively and effectively block the interaction between the ligand-bound ER and the cognate ER-coregulatory protein in breast cancer cells. This discovery has been exploited to develop the present disclosure, which includes novel compounds and therapeutic compositions, methods for inhibiting androgen signaling, methods for inhibiting estrogen signaling, methods for inhibiting the interaction between a co-regulatory protein and an androgen or estrogen receptor, and methods for treating cancer.

One aspect of the present disclosure is directed to novel compounds of Formula (I),

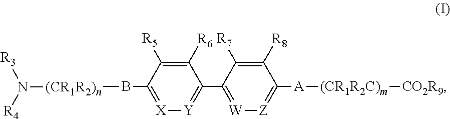

and pharmaceutically acceptable salts, hydrates, solvates, tautomers, and prodrugs thereof,

wherein: A and B are each independently a bond or —O—; R₁ and R₂ are each independently H or alkyl; R₃ and R₄ are each independently H, alkyl, cycloalkyl, aralkyl —C(O)alkyl, —C(O)NH(alkyl), —C(O)N(alkyl)₂; R₅, R₆, R₇, and R₈ are each independently H, alkyl, aralkyl, or cycloalkyl; n and in are each independently an integer from 1-6; W, X, Y, and Z are each independently CH or N; and R₉ is H, alkyl, aralkyl, —NH-alkyl, —N(alkyl)₂, —NH-aminoacid, or azole.

Another aspect of the present disclosure is directed to novel compounds of Formula (IA),

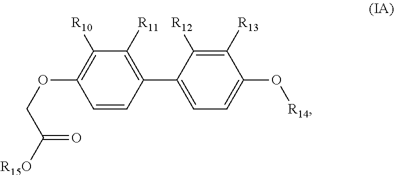

and pharmaceutically acceptable salts, hydrates, solvates, tautomers, and prodrugs thereof,

wherein: R₁₀, R₁₁, R₁₂, and R₁₃ are each independently H, C₁-C₄alkyl, or C₁-C₄alkyl-aryl; R₁₄ is H or C₁-C₆alkyl-di(C₁-C₄)alkylamino; and R₁₅ is H or C₁-C₄alkyl.

In some embodiments, R₁₀ is isopropyl, sec-butyl, tert-butyl, or benzyl. In other embodiments, R₁₁ is isopropyl, sec-butyl, tert-butyl, or benzyl. In still other embodiments, R₁₂ is isopropyl, sec-butyl, tert-butyl, or benzyl. In certain embodiments, R₁₃ is isopropyl, sec-butyl, tert-butyl, or benzyl. In yet other embodiments, R₁₅ is ethyl.

In further embodiments, R₁₄ is

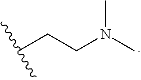

In each embodiment, the compound may be a pharmaceutically acceptable salt, hydrate, solvate, tautomer, or prodrug.

Another aspect of the disclosure is directed to a method of disrupting androgen signaling. In this method a sample is obtained that contains an androgen receptor and a co-regulatory protein. The sample is contacted with a compound of Formula (I) or (IA), or a pharmaceutically acceptable salt, hydrate, solvate, tautomer, or prodrug of Formula (I) or (IA). Next, an amount of co-regulatory protein-androgen receptor complex formed is detected. Then, the amount of complex formed in the sample is compared to an amount of complex formed in a control sample that does not contain the compound of Formula (I) or (IA). The androgen signaling is disrupted when the amount of complex in the sample is detectably less than the amount of complex formed in the control sample.

In some embodiments of the method, R₁₀ or R₁₁ of Formula (IA) is isopropyl, sec-butyl, or tert-butyl. In other embodiments R₁₀ or R₁₁ is benzyl. In further embodiments, R₁₂ or R₁₃ of Formula (IA) is isopropyl, sec-butyl, or tert-butyl. In still other embodiments, R₁₂ or R₁₃ is benzyl.

In certain embodiments, R₁₄ of Formula (IA) is

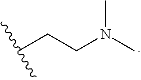

In particular embodiments, R₁₅ of Formula (IA) is ethyl.

Another aspect of the disclosure is directed to a method of disrupting estrogen signaling. In this method a sample is obtained that contains an estrogen receptor and a co-regulatory protein. The sample is contacted with a compound of Formula (I) or (IA), or a pharmaceutically acceptable salt, hydrate, solvate, tautomer, or prodrug of Formula (I) or (IA). Next, an amount of co-regulatory protein-estrogen receptor complex formed is detected. Then, the amount of complex formed in this sample is compared to an amount of complex formed in a control sample that does not contain the compound of Formula (I) or (IA). The estrogen signaling is disrupted when the amount of complex in the sample is detectably less than the amount of complex formed in the control sample.

In some embodiments of the method, R¹⁰ or R¹¹ of Formula (IA) is isopropyl, sec-butyl, or tert-butyl. In other embodiments R₁₀ or R₁₁ is benzyl. In further embodiments, R₁₂ or R₁₃ of Formula (IA) is isopropyl, sec-butyl, or tert-butyl. In still other embodiments, R₁₂ or R₁₃ is benzyl.

In certain embodiments, R₁₄ of Formula (IA) is

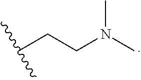

In particular embodiments, R₁₅ of Formula (IA) is ethyl.

Yet another aspect of this disclosure is directed to a method of inhibiting the interaction between a ligand-bound andogen receptor and a co-regulatory protein. In this method, a sample containing a ligand-bound androgen receptor and a co-regulatory protein is obtained. Then, the sample is contacted with a compound of Formula (I) or (IA), or pharmaceutically acceptable salt, hydrate, solvate, tautomer, or prodrug of Formula (I) or (IA). Next, an amount of co-regulatory protein-androgen receptor complex formed is detected. Then, the amount of complex formed is compared to an amount of complex formed in a control sample that does not contain the compound of Formula (I) or (IA). The interaction between a ligand-bound andogen receptor and a co-regulatory protein is inhibited when the amount of complex in the sample is detectably less than the amount of complex formed in the control sample.

In some embodiments of the method, R₁₀ or R₁₁ of Formula (IA) is isopropyl, sec-butyl, or tert-butyl. In other embodiments R₁₀ or R₁₁ is benzyl. In further embodiments, R₁₂ or R₁₃ of Formula (IA) is isopropyl, sec-butyl, or tert-butyl. In still other embodiments, R₁₂ or R₁₃ is benzyl.

In certain embodiments, R₁₄ of Formula (IA) is

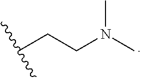

In particular embodiments, R₁₅ of Formula (IA) is ethyl.

Yet a further aspect of this disclosure is directed to a method of inhibiting the interaction between a ligand-bound estrogen receptor and a co-regulatory protein. In this method, a sample containing a ligand-bound estrogen receptor and a co-regulatory protein is obtained. Then, the sample is contacted with a compound of Formula (I) or (IA), or pharmaceutically acceptable salt, hydrate, solvate, tautomer, or prodrug of Formula (I) or (IA). Next, an amount of co-regulatory protein-estrogen receptor complex formed is detected. Then, the amount of complex formed in the sample is compared to an amount of complex formed in a control sample that does not contain the compound of Formula (I) or (IA). The interaction between a ligand-bound estrogen receptor and a co-regulatory protein is inhibited when the amount of complex in the sample is detectably less than the amount of complex formed in the control sample.

In some embodiments of the method, R₁₀ or R₁₁ of Formula (IA) is isopropyl, sec-butyl, or tert-butyl. In other embodiments R₁₀ or R₁₁ is benzyl. In further embodiments, R₁₂ or R₁₃ of Formula (IA) is isopropyl, sec-butyl, or tert-butyl. In still other embodiments, R₁₂ or R₁₃ is benzyl.

In certain embodiments, R₁₄ is

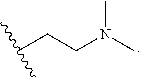

In particular embodiments, R₁₅ is ethyl.

A further aspect of this disclosure is directed to a method of inhibiting cell proliferation in cancer cells. In this method, a sample containing a cancer cell is contacted with a compound of Formula (I) or (IA), or a pharmaceutically acceptable salt, hydrate, solvate, tautomer, or prodrug of Formula (I) or (IA). Then, the proliferative state of the cell in the sample is detected. Cell stasis or death detected in the sample indicates the inhibition of cell proliferation.

In some embodiments of the method, R₁₀ or R₁₁ of Formula (IA) is isopropyl, sec-butyl, or tert-butyl. In other embodiments R₁₀ or R₁₁ is benzyl. In further embodiments, R₁₂ or R₁₃ of Formula (IA) is isopropyl, sec-butyl, or tert-butyl. In still other embodiments, R₁₂ or R₁₃ is benzyl.

In certain embodiments, R₁₄ is

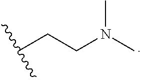

In particular embodiments, R₁₅ is ethyl.

One aspect of this disclosure is directed to a method of treating cancer in a subject. In this method, a subject is administered a therapeutically effective amount of a compound of Formula (I) or (IA), or a pharmaceutically acceptable salt, hydrate, solvate, tautomer, or prodrug of Formula (I) or (IA). Then, a decrease in a symptom of the cancer is detected. The compound treats the cancer by inhibiting androgen or estrogen signaling in the cancer.

In some embodiments, the cancer is prostate cancer. In other embodiments, the cancer is breast cancer.

In some embodiments of the method, R₁₀ or R₁₁ of Formula (IA) is isopropyl, sec-butyl, or tert-butyl. In other embodiments R₁₀ or R₁₁ is benzyl. In further embodiments, R₁₂ or R₁₃ of Formula (IA) is isopropyl, sec-butyl, or tert-butyl. In still other embodiments, R₁₂ or R₁₃ is benzyl.

In certain embodiments, R₁₄ is

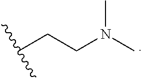

In particular embodiments, R₁₅ is ethyl.

### BRIEF DESCRIPTION OF THE DRAWINGS

The foregoing and other objects of this disclosure, the various features thereof, as well as the invention itself may be more fully understood from the following description, when read together with the accompanying drawings in which:

FIG. 1A is a graphic representation of the results of the In Vitro Coactivator Binding Inhibitor TR-FRET CBI Assay with ERα.

FIG. 1B is a graphic representation of the results of the In Vitro Coactivator Binding Inhibitor TR-FRET CBI Assay.

FIG. 2A is a graphic representation of the Cell-Based Coactivator Binding Inhibitor Reporter Gene Assay.

FIG. 2B is a graphic representation of the Cell-Based Coactivator Binding Inhibitor Reporter Gene Assay.

FIG. 3 is a graphic representation of the results of the mammalian two-hybrid assay.

FIG. 4 is graphic representation of a second mammalian two hybrid assay performed to determine the proper dose for use in cell culture.

FIG. 5 is graphic representation of a quantitative-PCR analysis of ERα target genes.

FIG. 6 is a graphic representation of the results of an RBA assay.

### DETAILED DESCRIPTION

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications and this disclosure.

This disclosure relates to novel compounds, pharmaceutical compositions comprising these compounds, methods for inhibiting androgen signaling, methods for inhibiting estrogen signaling, methods for inhibiting the interaction between a co-regulatory protein and an androgen or estrogen receptor, and methods for treating cancer.

### 1. DEFINITIONS

For convenience, certain terms employed in the specification, examples and claims are collected here. Unless defined otherwise, all technical and scientific terms used in this disclosure have the same meanings as commonly understood by one of ordinary skill in the art to which this disclosure belongs. The initial definition provided for a group or term provided in this disclosure applies to that group or term throughout the present disclosure individually or as part of another group, unless otherwise indicated.

The compounds of this disclosure include any and all possible isomers, stereoisomers, enantiomers, diastereomers, tautomers, pharmaceutically acceptable salts, and solvates thereof. Thus, the terms “compound” and “compounds” as used in this disclosure refer to the compounds of this disclosure and any and all possible isomers, stereoisomers, enantiomers, diastereomers, tautomers, pharmaceutically acceptable salts, and solvates thereof.

In general, the compositions of the disclosure can be alternately formulated to comprise, consist of, or consist essentially of, any appropriate components disclosed in this disclosure. The compositions of the disclosure can additionally, or alternatively, be formulated so as to be devoid, or substantially free, of any components, materials, ingredients, adjuvants or species used in the prior art compositions or that are otherwise not necessary to the achievement of the function and/or objectives of the present disclosure.

The articles “a” and “an” are used in this disclosure to refer to one or more than one (i.e., to at least one) of the grammatical object of the article. By way of example, “an element” means one element or more than one element.

The term “or” is used in this disclosure to mean, and is used interchangeably with, the term “and/or,” unless indicated otherwise.

The term “about” is used in this disclosure to mean a value − or + 20% of a given numerical value. Thus, “about 60%” means a value between 60-20% of 60 and 60+20% of 60 (i.e., between 48% and 72%).

The terms “alkyl” and “alk”, unless otherwise specifically defined, refer to a straight or branched chain alkane (hydrocarbon) radical, which may be fully saturated, mono- or polyunsaturated, and can include divalent radicals, having from 1 to about 15 carbon atoms. Examples of saturated hydrocarbon radicals include, but are not limited to, groups such as methyl (Me), ethyl (Et), n-propyl, isopropyl, n-butyl, t-butyl, isobutyl, sec-butyl, homologs and isomers of, for example, n-pentyl, n-hexyl, n-heptyl, n-octyl, 1,1-dimethyl-heptyl, 1,2-dimethyl-heptyl, and the like. An unsaturated alkyl group includes one or more double bonds, triple bonds or combinations thereof. Examples of unsaturated alkyl groups include but are not limited to, vinyl, propenyl, crotyl, 2-isopentenyl, allenyl, butenyl, butadienyl, pentenyl, pentadienyl, 3-(1,4-pentadienyl), hexenyl, hexadienyl, ethynyl, propynyl, butynyl, and higher homologs and isomers. The term “C₁₋ₘ-alkyl” refers to an alkyl having from 1 to about m carbon atoms. The alkyl group may be optionally substituted with one or more substituents, e.g., 1 to 5 substituents, at any available point of attachment, as defined below.

The term “aralkyl”, unless otherwise specifically defined, refers to an alkyl group where an H has been replaced with an aryl group.

The term “aryl”, unless otherwise specifically defined, refers to cyclic, aromatic hydrocarbon groups that have 1 to 5 aromatic rings, including monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The aryl group may be optionally substituted by one or more substituents, e.g., 1 to 5 substituents, at any point of attachment. In addition to the substituents described under the definition of “substituted,” other exemplary substituents include, but are not limited to, nitro, cycloalkyl or substituted cycloalkyl, cycloalkenyl or substituted cycloalkenyl, cyano, alkyl, fused cyclic groups, fused cycloalkyl, fused cycloalkenyl, fused heterocycle, and fused aryl, and those groups recited above as exemplary alkyl substituents. The substituents can themselves be optionally substituted.

The term “azole”, unless otherwise specifically defined, is a five-membered nitrogen heterocyclic ring containing at least one other noncarbon atom, e.g., nitrogen, sulfur or oxygen. The azole can be substituted or unsubstituted.

The term “cycloalkyl”, unless otherwise specifically defined, refers to a saturated or partially saturated cyclic hydrocarbon group containing from 1 to 4 rings and 3 to 8 carbons per ring, including, for example, 4 to 7 membered monocyclic groups, 7 to 12 membered bicyclic groups, or 8 to 16 membered tricyclic ring systems, polycyclic groups, or bridged systems. Exemplary groups include, but are not limited to, cyclopropyl, cyclobutyl, cyclopentyl, cyclohexyl, cycloheptyl, adamantyl, cyclobutenyl, cyclopentenyl, and cyclohexenyl, etc. The cycloalkyl group may be optionally substituted with one or more substituents, e.g., 1 to 5 substituents, at any available point of attachment. In addition to the substituents described under the definition of “substituted,” other exemplary substituents include, but are not limited to, nitro, cyano, alkyl, spiro attached or fused cyclic substituents, spiro-attached cycloalkyl, spiro-attached cycloalkenyl, spiro-attached heterocycle, fused cycloalkyl, fused cycloalkenyl, fused heterocycle, fused aryl, and those groups recited above as exemplary alkyl substituents. The substituents can themselves be optionally substituted. The term “divalent cycloalkyl radicals” unless otherwise specifically defined refers to the general formula: -cycloalkyl-.

The term “adamantyl”, unless otherwise specifically defined, includes, but is not limited to, 1 adamantyl, 2 adamantyl, and 3 adamantyl. The adamantyl group may be optionally substituted with the groups recited as exemplary cycloalkyl substituents as well as the substituents described under the definition of “substituted.”

The term “dialkylamino”, unless otherwise specifically defined, refers to the general formula —N(alkyl)₂. For example, dialkyl(C₂-C₃)amino refers to the general formulae —N(CH₂CH₃)₂ or —N(CH₂CH₂CH₃)₂. Unless otherwise specifically limited dialkylamino includes cyclic amine compounds such as piperidine and morpholine.

The terms “heterocycle” and “heterocyclic”, unless otherwise specifically defined, refer to fully saturated, or partially or fully unsaturated, including aromatic (i.e., “heteroaryl”) cyclic groups (for example, 4 to 7 membered monocyclic, 7 to 12 membered bicyclic, or 8 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached to the remainder of the molecule at any heteroatom or carbon atom of the ring or ring system. Exemplary monocyclic heterocyclic groups include, but are not limited to, azetidinyl, pyrrolidinyl, pyrrolyl, pyrazolyl, oxetanyl, dioxanyl, dioxolanyl, oxathiolanyl, pyrazolinyl, imidazolyl, imidazolinyl, imidazolidinyl, oxazolyl, oxazolidinyl, isoxazolinyl, isoxazolyl, thietanyl, azetidine, diazetidine, thiolanyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, purinyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include, but are not limited to, indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include, but are not limited to, carbazolyl, benzidolyl, phenanthrolinyl, acridinyl, phenanthridinyl, xanthenyl, and the like.

A heterocyclic group may be optionally “substituted” with one or more substituents, e.g., 1 to 5 substituents, at any available point of attachment. In addition to the substituents described under the definition of “substituted,” other exemplary substituents include, but are not limited to, cycloalkyl or substituted cycloalkyl, cycloalkenyl or substituted cycloalkenyl, nitro, oxo (i.e., ═O), cyano, alkyl or substituted alkyl, spiro-attached or fused cyclic substituents at any available point or points of attachment, spiro-attached cycloalkyl, spiro-attached cycloalkenyl, spiro-attached heterocycle (excluding heteroaryl), fused cycloalkyl, fused cycloalkenyl, fused heterocycle, fused aryl, and the like. The substituents can themselves be optionally substituted.

The term “substituted” means substituted by a below-described substituent group in any possible position. Substituent groups for the above moieties useful in this disclosure are those groups that do not significantly diminish the biological activity of the disclosed compound. Substituent groups that do not significantly diminish the biological activity of the disclosed compound include, but are not limited to, H, halogen, N₃, NCS, CN, NO₂, NX₁X₂, OX₃, C(X₃)₃, OAc, O-acyl, O-aroyl, NH-acyl, NH-aroyl, NHCOalkyl, CHO, C(halogen)₃, Ph, OPh, CH₂Ph, OCH₂Ph, COOX₃, SO₃H, PO₃H₂, SO₂NX₁X₂, CONX₁X₂, alkyl, alcohol, alkoxy, dioxolanyl, alkylmercapto, dithiolanyl, dithianyl, alkylamino, dialkylamino, sulfonamide, thioalkoxy or methylene dioxy when the substituted structure has two adjacent carbon atoms, wherein Xᵢ and X₂ each independently comprise H or alkyl, and X₃ comprises H, alkyl, hydroxyloweralkyl. Unless otherwise specifically limited, a substituent group may be in any possible position.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The phrase “pharmaceutically acceptable” is employed in this disclosure to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “salt” or “salts”, as employed in this disclosure, denote acidic and/or basic salts formed with inorganic and/or organic acids and bases.

The term “treating” with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject. The actual amount which comprises the “effective amount” or “therapeutically effective amount” will vary depending on a number of conditions including, but not limited to, the particular disorder being treated, the severity of the disorder, the size and health of the patient, and the route of administration. A skilled medical practitioner can readily determine the appropriate amount using methods known in the medical arts.

As used in this disclosure, the term “subject” includes, without limitation, a human or an animal. Exemplary animals include, but are not limited to, mammals such as mouse, rat, guinea pig, dog, cat, horse, cow, pig, monkey, chimpanzee, baboon, or rhesus monkey.

The terms “administer”, “administering”, or “administration” as used in this disclosure refer to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a compound of Formula (I).

The terms “isolated” and “purified” as used in this disclosure refer to a component separated from other components of a reaction mixture or a natural source. In certain embodiments, the isolate contains at least about 50%, at least about 55%, at least about 60%, at least about 65%, at least about 70%, at least about 75%, at least about 80%, at least about 85%, at least about 90%, at least about 95%, or at least about 98% of the compound or pharmaceutically acceptable salt of the compound by weight of the isolate.

The term “tautomer” as used in this disclosure refers to compounds produced by the phenomenon wherein a proton of one atom of a molecule shifts to another atom. (March, Advanced Organic Chemistry: Reactions, Mechanisms and Structures, 4th Ed., John Wiley & Sons, pp. 69-74 (1992)).

The following abbreviations are used in this disclosure and have the following definitions: DMF is dimethylformamide; DMSO is dimethylsulfoxide; THF is tetrahydrofuran; Tris is tris(hydroxymethyl)aminomethane; “h” is hour or hours; and “RT” is room temperature.

### 2. COMPOUNDS

This disclosure provides the useful compounds that are disruptors of co-activator binding and can be characterized by having some or all of the following properties. First, the compounds can have drug-like properties, i.e., low molecular weight and restricted conformational mobility and modest lipophilicity. Second, the compounds contain internal functional groups within an α-helix-like structure that would mimic the lipophilic leucine (L) residues to effect appropriate interactions with the NRB-binding site. Third, the compounds have functional groups at the termini to interact with the “charge clamp” residues. Fourth, the compounds are amenable to elaboration to include functional groups that would resemble the flanking amino acid residues of the co-activator proteins.

The present disclosure provides novel compounds of Formula

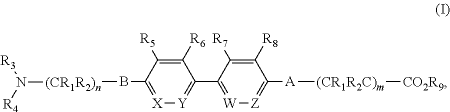

and pharmaceutically acceptable salts, hydrates, solvates, tautomers, and prodrugs thereof,

wherein A, B, R₁-R₉, n, m, W, X, Y, and Z are as described above for Formula (I).

Nonlimiting illustrative compounds of Formula (I) include:

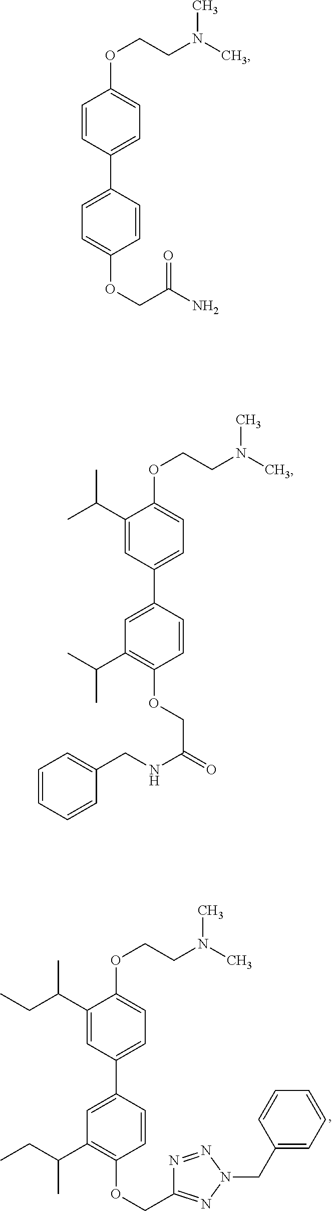

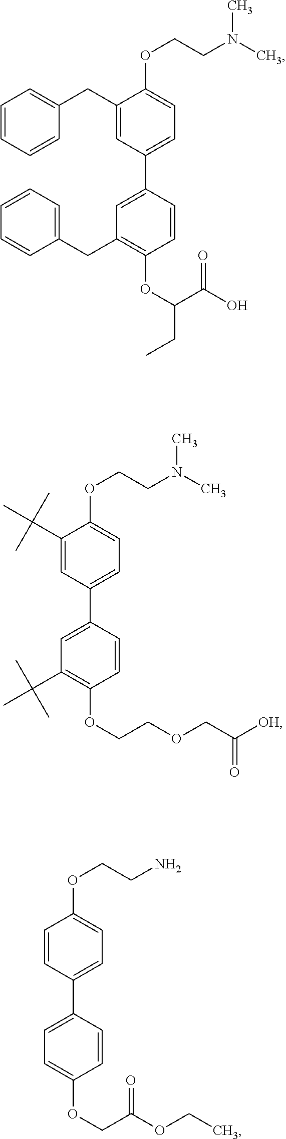

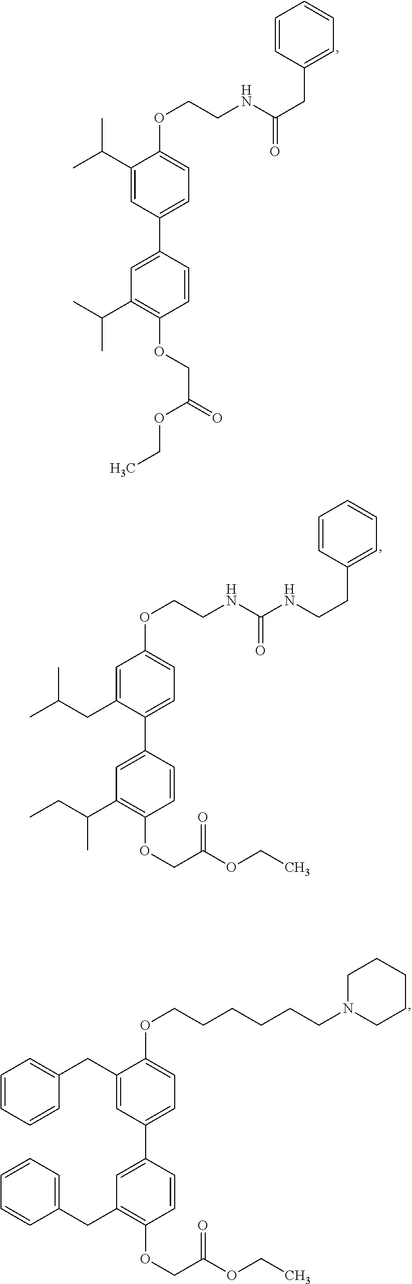

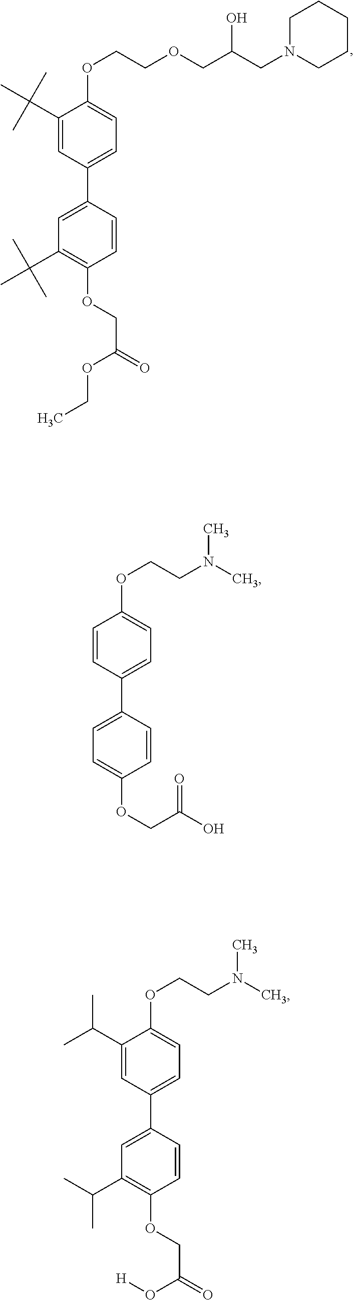

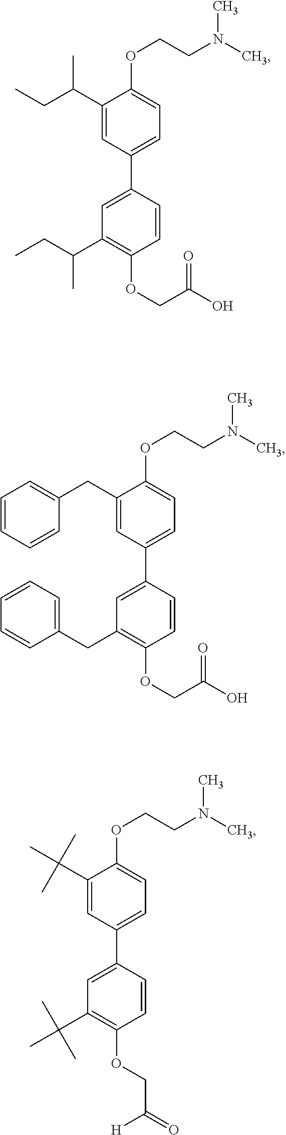

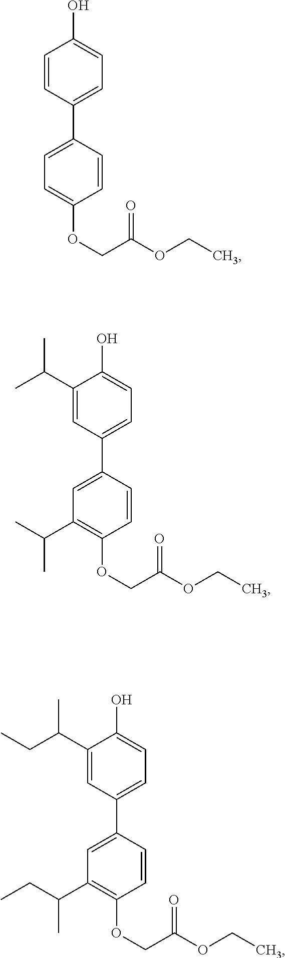

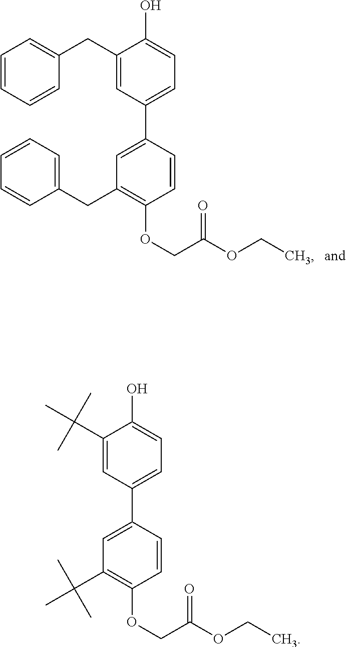

The present disclosure also provides novel compounds of Formula (IA),

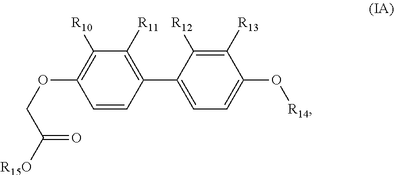

and pharmaceutically acceptable salts, hydrates, solvates, tautomers, and prodrugs thereof,

wherein R₁₀-R₁₅ are as described above for Formula (IA).

Nonlimiting illustrative compounds of Formula (IA) include:

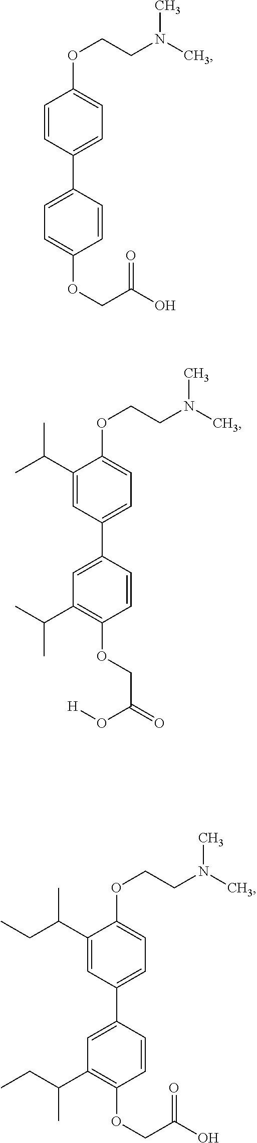

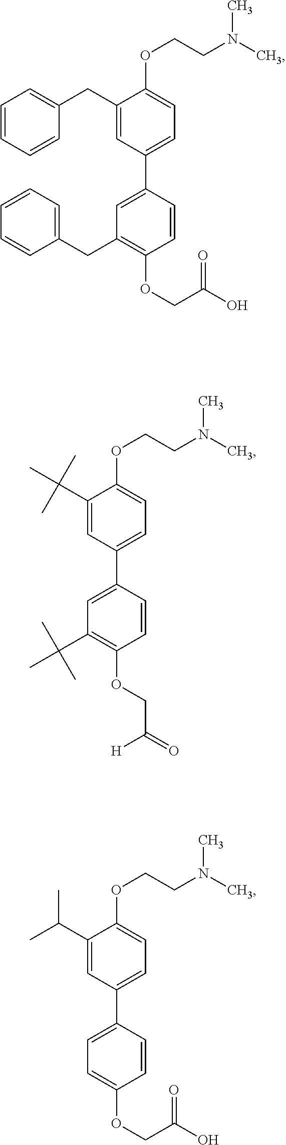

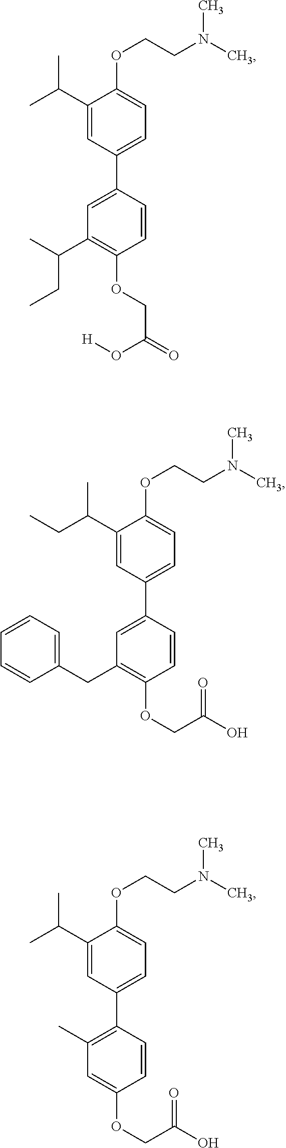

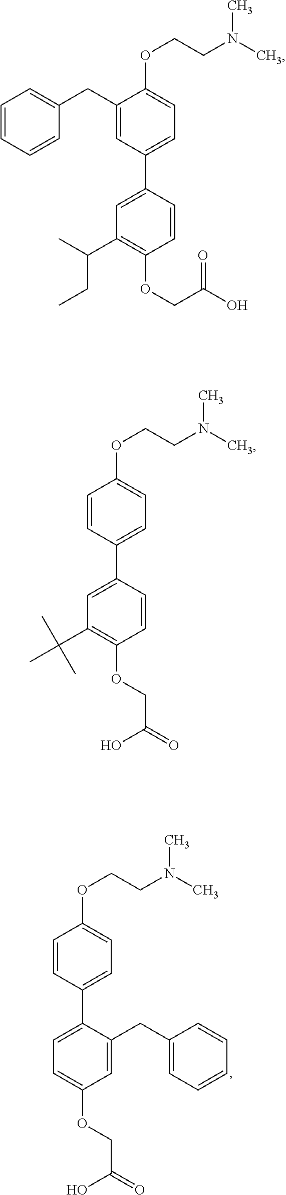

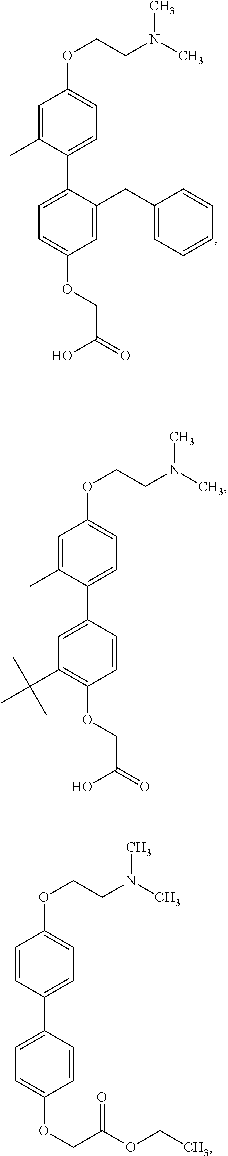

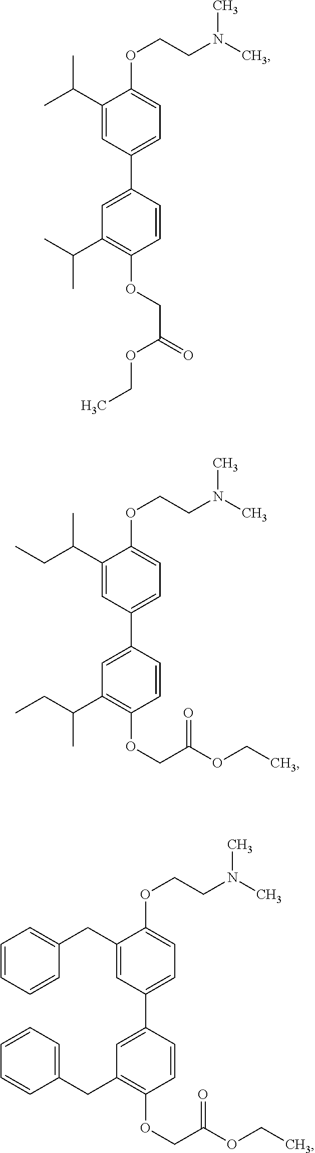

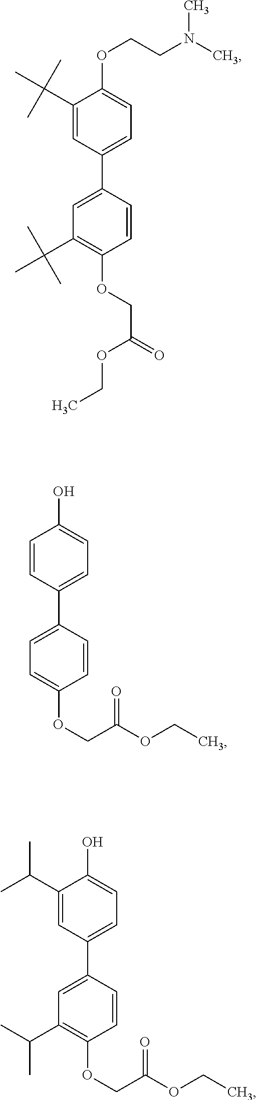

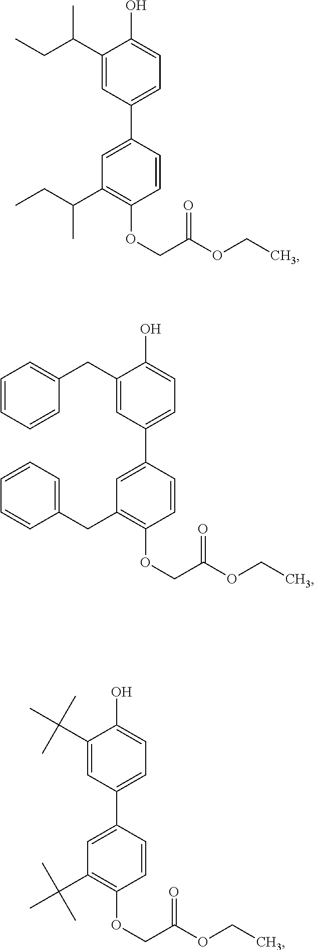

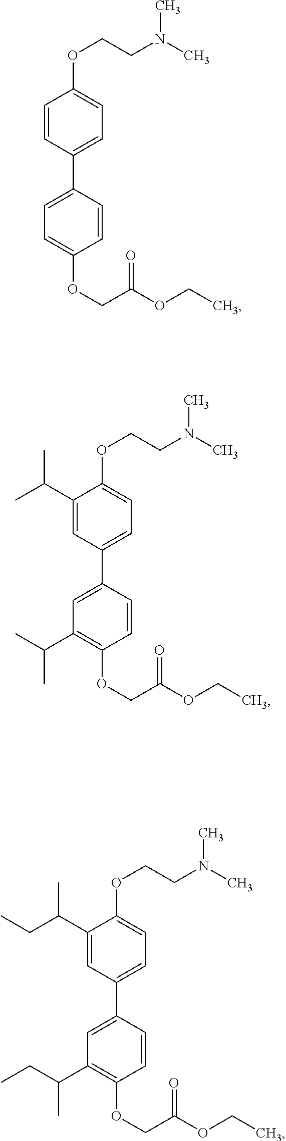

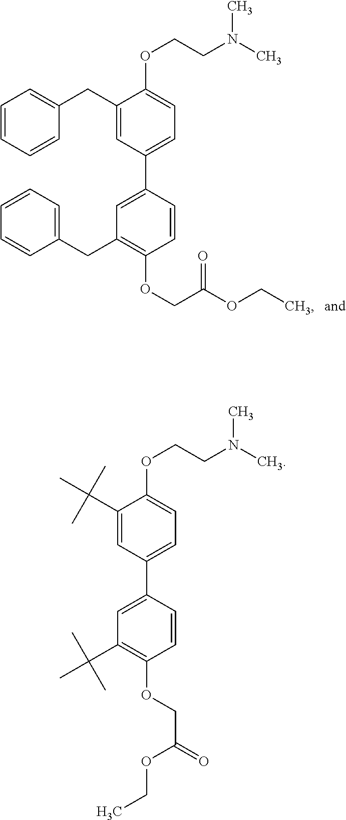

The compounds of Formula (I) or (IA) can also form salts which are also within the scope of this disclosure. Reference to a compound of the present disclosure is understood to include reference to salts thereof, unless otherwise indicated. The compounds of Formula (I) or (IA) may form pharmaceutically acceptable (i.e., non-toxic, physiologically acceptable) salts as well as other salts that are also useful, e.g., in isolation or purification steps which can be employed during preparation.

The compounds of Formula (I) or (IA) which contain a basic moiety, such as, but not limited to, an amine or a pyridine or imidazole ring, can form salts with a variety of organic and inorganic acids. Exemplary acid addition salts include, but are not limited to, acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of Formula (I) or (IA) which contain an acidic moiety, such as, but not limited to, a carboxylic acid, can form salts with a variety of organic and inorganic bases. Exemplary basic salts include, but are not limited to, ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups can be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and the like.

Exemplary nonlimiting compounds of Formula (I) and (IA) are listed in the Examples section below. Solvates of the compounds of this disclosure, including hydrates of the compounds, as well as mixtures of the hydrate- and the keto-form of the compounds, are within the scope of this disclosure.

### 3. METHODS OF MAKING THE SUBSTITUTED BIPHENYLS

The novel compounds can be synthesized by chemical means as described in the following generic schemes and in the Examples below. Novel compounds may be synthesized from commercially available starting material. The compounds need not be made exclusively by the illustrative syntheses. A person of skill in the art understands that additional methods of making the compounds exist. A person of skill in the art also understands that additional general synthetic schemes for the compounds disclosed herein can be understood from the illustrative schemes below.

A series of bi-aryl, mixed bi-aryl-heteroaryl, and bi-heteroaryl compounds can be synthesized using a convergent approach, i.e., preparing the amino and carboxylic acid units separately and ligating them at the end. Although the schemes exemplify the use of phenyl, other aryls and heteroaryls can be also used and the chemistry adjusted accordingly by means available to one skilled in the art.

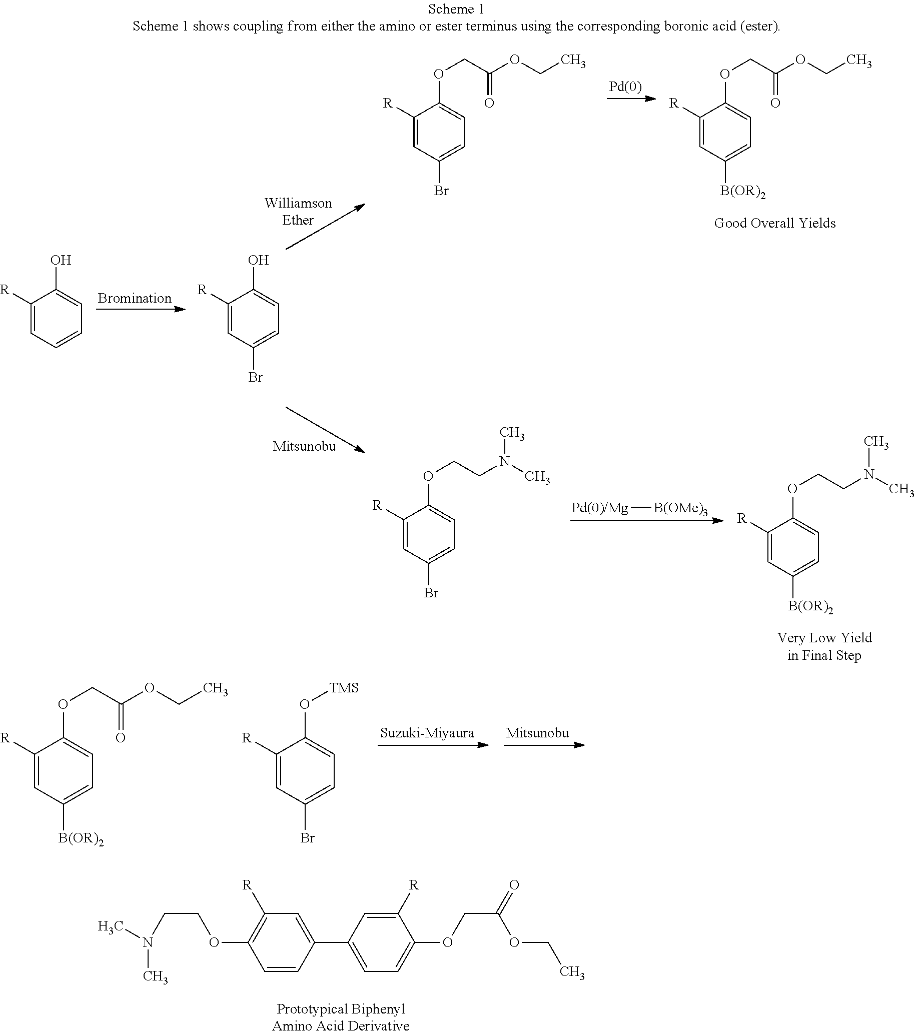

The 4-bromophenyl ethers can be prepared by boronation of the dimethylaminoethoxyphenyl bromide using either Pd(0) catalysts or Grignard reagents. Alternatively, the Suzuki coupling reaction can also be done with the unsubstituted dimethylaminoethoxyphenyl bromide. Alternatively, a scheme including the initial bromination of 2-(3-)substituted phenols was used, as described below. The carboxylic acid terminus was introduced by attachment of an α-bromo-alkanoate ester (Williamson ether synthesis) followed by conversion to the boronate ester (Suzuki-Miyaura reaction).

Substituted biphenyl derivatives are also prepared starting from 2- and 3-substituted phenols, including where R₁ and/or R₂ in Formula (I) are each independently methyl, isopropyl, tert-butyl, benzyl, and trifluoromethyl. These substituents mimic the side chains of alanine, leucine, isoleucine (valine), phenylalanine, and the unnatural amino acid trifluoroalanine. Bromination with tetrabutylammonium tribromide provides the 4-bromo-phenols. For trifluoromethyl phenol bromine is used in acetic acid. Alkylation with sodium hydride-ethyl bromoacetate followed by Pd(0)-catalyzed borylation gives the carboxy-terminated unit.

The 4-bromo-2-substituted phenol is protected as the TMS-ether to generate the precursor for the amino-terminated unit. Suzuki coupling of the two components, followed by desilylation and Mitsunobu coupling with the amino alcohol (R₃ from Formula (I) can be methyl, tetramethylene) gives the penultimate product. Hydrolysis of the ethyl ester then provides the target amino-carboxy substituted biphenyls. The synthetic chemistry associated with the 3-substituted phenols is the same as for the 2-substituted phenols, with the possible exception for the initial 4-bromination. The scheme allows to prepare a variety of biphenyl derivatives in which the functional side chain groups can be widely varied, e.g., symmetrically or asymmetrically. All new compounds are characterized by NMR, HPLC and MS. Iridium catalysts are used for the direct introduction of boronate esters onto the non-brominated phenols or preparation of phenols from meta substituted benzenes, or arylacetic acids/phenylethyl amines, is also useful as starting material. Replacement of the phenyl by pyridyl ring may affect the introduction of the alkyl/aralkyl side chains but may not affect the introduction of the acid/base termini or the biaryl couplings. Deletion of the oxy-group in the termini could also affect the introduction of the side chains and 4-bromo groups, but not the coupling process.

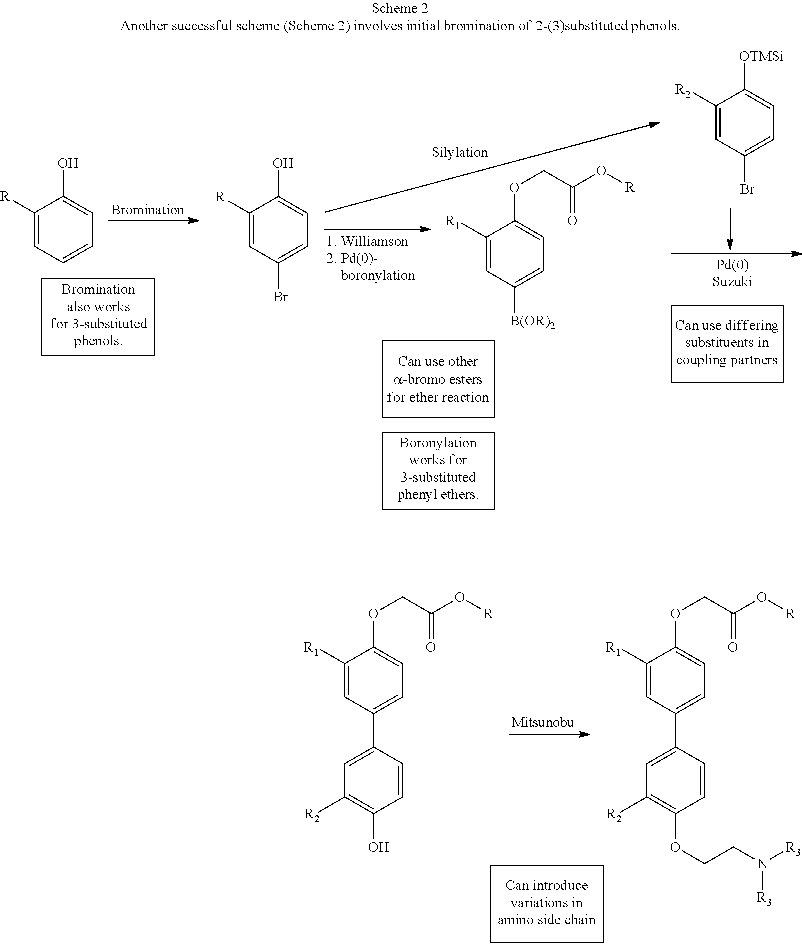

The carboxylic acid terminus is introduced by attachment of an α-bromo-alkanoate ester (Williamson ether synthesis) followed by conversion to the boronate ester (Suzuki-Miyaura reaction). The phenolic group was protected as a silyl ether, before performing the coupling of the aminoalkoxyphenyl bromide to the ester unit; removed the silyl group, and attached the amino-alkoxy group using the Mitsunobu reaction in a strategy similar to that used by Scanlan for TRα agonists.

Three Series of 4,4′-Disubstituted Biphenyl Derivatives

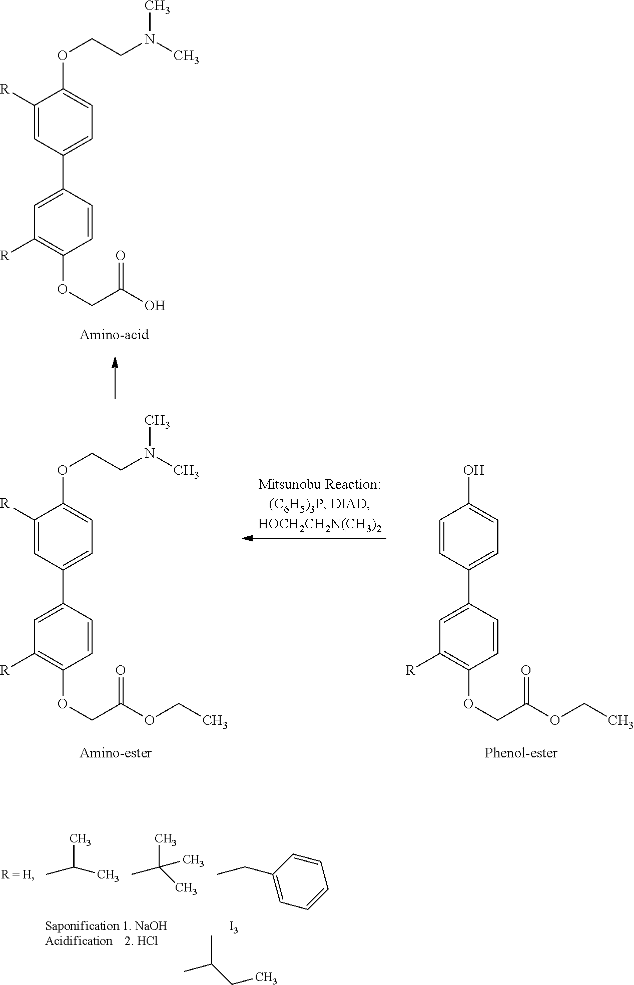

From the intermediate phenol-ester, introduction of the amino terminus is achieved using the Mitsunobu reaction with the alcohol (or alternatively using sodium hydride and the chloroethyldimethylamine hydrochloride). The resulting amino-ester derivative is converted to the corresponding amino-acid product using sodium hydroxide saponification. Addition of hydrogen chloride yields the amino-acid as the zwitterions (neutral) or if in excess, the hydrochloride salt.

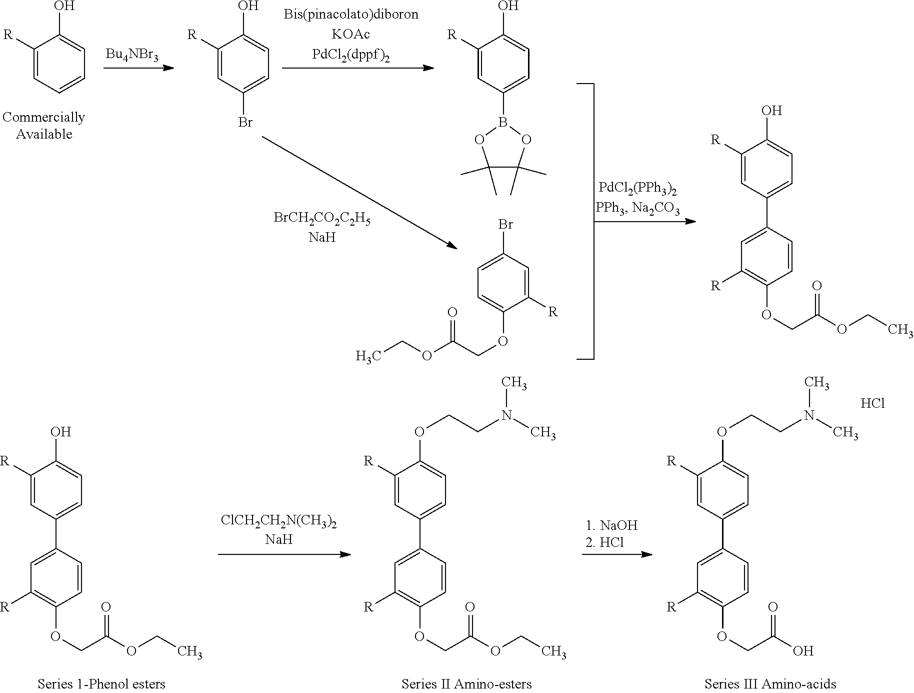

From commercially available 2-(ortho)substituted phenols, introduction of the 4-bromo group is achieved in high yields using brominating agents, such as tetrabutylammonium tribromide. Other brominating agents, such as bromine, N-bromosuccinimide, pyridinium tribromide, are equally effective. Miyaura coupling of the 4-bromo intermediate with bis(pinacolato)diboron using PdCl₂(dppf)₂ as the catalyst and potassium acetate as the base gave the requisite 4-boronate ester. The same intermediate was converted to the corresponding phenyl ether using ethyl 2-bromoacetate and sodium hydride. Suzuki coupling of the two intermediates was achieved using PdCl₂[P(C₆H₅)₃]₂ as the catalyst and sodium carbonate as the base. Chromatographic separation on silica gel gave the pure derivative [Series I]. Conversion to the amino-ester series was achieved using the Williamson ether synthesis, in this case, using sodium hydride and chloroethyldimethyl amine hydrochloride. Chromatographic separation on silica gel gave pure products [Series II]. Saponification with sodium hydroxide followed by acidification gave the hydrochlorides that were purified by crystallization [Series III].

### Preparation of 4-bromo-2-substituted Phenols

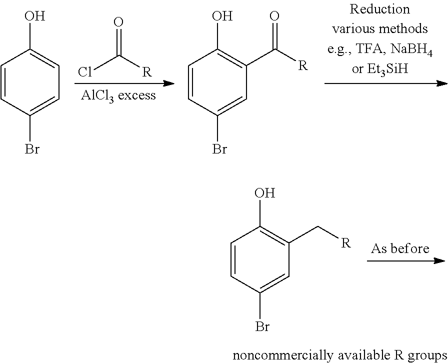

### Preparation of 4-bromo-3-substituted Phenols

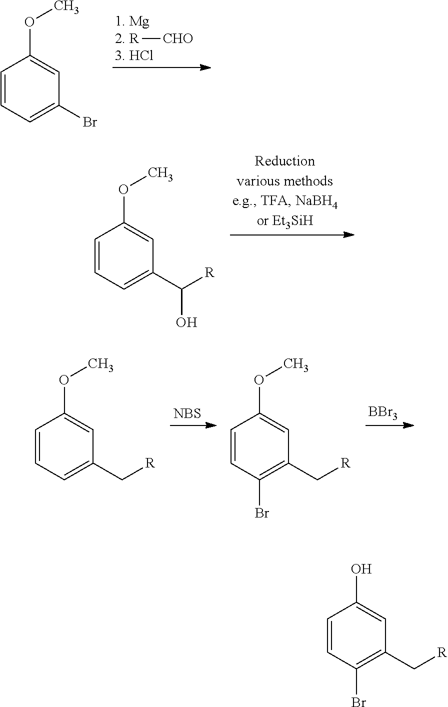

Preparation of the non-commercially available 2-substituted-4-bromophenols is achieved by using the Fries rearrangement. Acylation of the 4-bromophenol in the presence of a Lewis acid, such as aluminum trichloride (or boron trifluoride), gives the O-acylated material which rearranges upon heating to the more stable C-(2)-acyl product. [The presence of the bromine in the 4-position prevents migration to that more favorable acylation site.] Reduction of the carbonyl is then achieved by a number of mild methods, including trifluoroacetic acid-sodium borohydride, or triethylsilane. Stronger reducing agents or catalytic hydrogenation would remove the 4-bromo-substituent. At this point, the compounds are introduced back into the previously described synthetic pathways.

Preparation of non-commercially available 3-substituted phenols begins with commercially available 3-bromo-anisole. Generation of the Grignard reagent with magnesium, followed by addition of commercially available aldehydes gives, upon workup, the benzylic alcohols. Reduction with a variety of reducing reagents, including trifluoroacetic acid-sodium borohydride, triethylsilane and catalytic hydrogenation gives the corresponding 3-substituted anisoles. Careful mono-bromination at the 4-position is achieved using N-bromosuccinimide. Chromatographic separation yields the pure isomer which is converted to the corresponding phenolic intermediate using boron tribromide. Chromatographic separation gives the pure intermediate which can be used in the previously described pathways.

### Preparation of 4-bromo-2,6-disubstituted Phenols

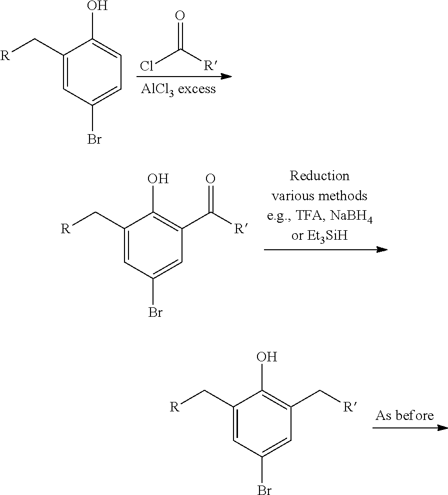

### Preparation of 4-bromo-2,5-disubstituted Phenols

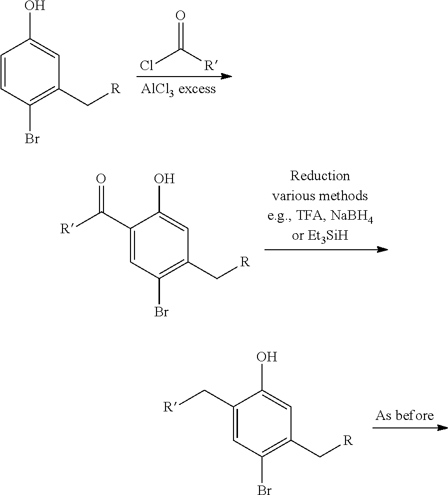

The 2,6-(ortho,ortho)-di-substituted material are obtained using the previously synthesized 2-substituted-4-bromophenols. Repetition of the Fries reaction, with the same or different acid chloride, using aluminum trichloride in a polyhalogenated solvent at elevated temperature gives the more stable 6-acylated intermediate. Reduction with mild reducing agents, such as trifluoroacetic acid-sodium borohydride, or triethylsilane, gives the 2,6-di-substituted intermediate. This intermediate can then undergo Miyaura reactions at the brominated site, or Williamson ether synthesis reactions/Mitsunobu reactions at the phenolic position as described in previous schemes.

The 2,5-(ortho,meta)-disubstituted bromophenols are obtained using the previously synthesized 3-substituted-4-bromophenols. Repetition of the Fries reaction, with the same or different acid chloride, using aluminum trichloride in a polyhalogenated solvent at elevated temperature gives the more stable 2-acylated intermediate. Reduction with mild reducing agents, such as trifluoroacetic acid-sodium borohydride, or triethylsilane, gives the 2,5-di-substituted intermediate. This intermediate can then undergo Miyaura reactions at the brominated site, or Williamson ether synthesis reactions/Mitsunobu reactions at the phenolic position as described in previous schemes.

Alternate Methods for Preparing Intermediates and Final Products

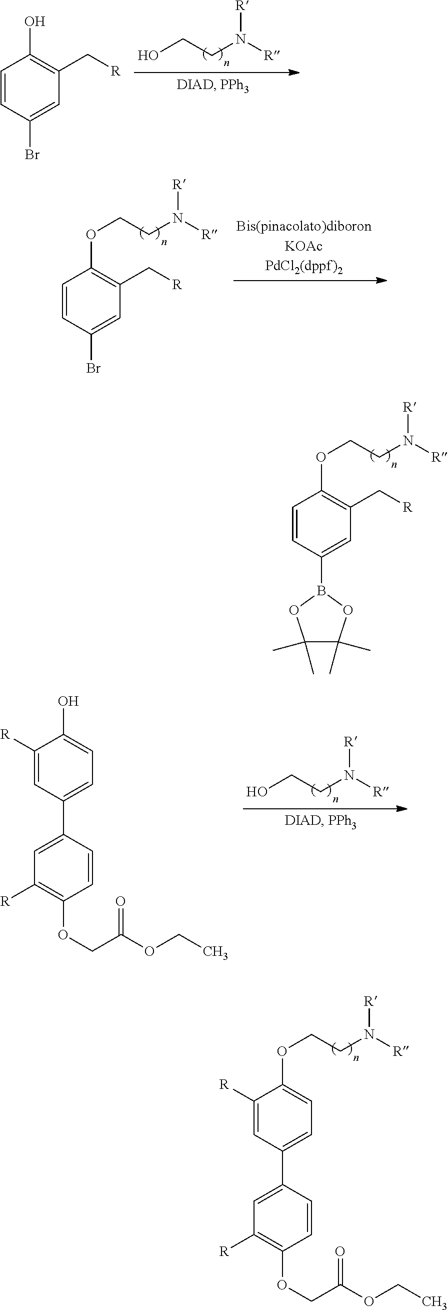

Variations in the dialkylaminoalkyl side chain are readily achieved using the Mitsunobu reaction. Reaction of the 2-(3-)-substituted-4-bromophenol with commercially available dialkylaminoalkanols, where R′ and R″ are hydrogen, alkyl, cycloalkyl, aralkyl, or heterocyclic, and where the alkyl chain can vary (n=1-10) and can contain heteroatoms, e.g., O, S, N. The Miyaura coupling with bis(pinacolato)diboron gives the corresponding 4-boronate ester that can be used for Suzuki coupling with appropriate 4-brominated phenols or 4-bromophenyl ethers. The Mitsunobu reaction that is used to generate the 4-bromo-phenyl ethers, can also be used to generate the corresponding series of disubstitutedaminoalkyl-biphenyl ethers shown in the lower reaction. The same substitution patterns are tolerated for this synthetic sequence.

Alternate Method for Making Amino-Phenols, Amino-Esters, Amino-Adds

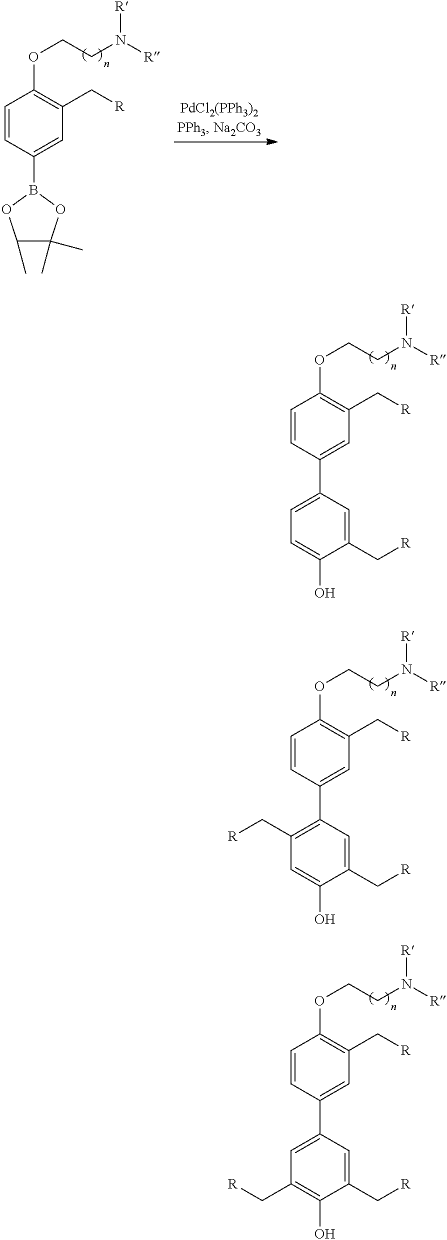

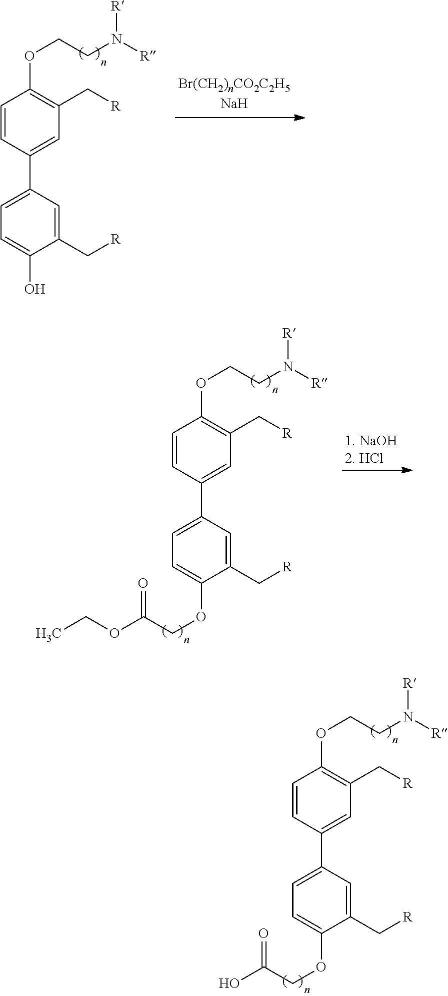

Using the variously substituted 4-bromo-phenyl ethers described in the previous schemes, one can do Suzuki coupling reactions with variously substituted 4-bromo-phenols bearing unsymmetrical substituents. The products can then undergo Williamson ether synthesis with, e.g., ethyl bromoacetate to give the amino-esters which can be converted, by saponification-acidification, to the amino acids. The sequence is not limited to alkyl bromoacetate—haloalkanoates, haloalkanoamides, haloalkanes, etc. can be used.

### 4. METHODS OF INHIBITION

This disclosure also provides methods for inhibiting androgen signaling, methods for inhibiting estrogen signaling, and methods for inhibiting the interaction between a co-regulatory protein and an androgen or estrogen receptor. The compounds of this disclosure are useful for these methods both in vivo and in vitro.

X-ray crystallographic studies of the liganded AR-LBD with NRB-containing peptides provided key information for this disclosure. Crystal structures of the AR-α-LBD that incorporated an NRB peptide were examined to generate a consensus binding site. Similarly, crystal structures of the ER-α-LBD that incorporated an NRB peptide were also examined to generate a consensus binding site. These data were then used to evaluate the relative binding properties of a series of compounds of this disclosure.

Co-crystallization studies using AR-LBD with androgenic agonists in the presence of peptides containing the LXXLL motif demonstrated that ligand binding causes the movement of helix-2 to generate a shallow groove/cleft in the receptor surface. Similarly, co-crystallization of ER-α-LBD with estrogenic agonists in the presence of peptides containing the LXXLL motif also demonstrated that ligand binding causes the movement of helix-2 to generate a shallow cleft in the receptor surface. In addition to presenting a lipophilic surface complementary to the leucine (L) side chains, the groove also contains a “charge clamp,” formed by lysine and glutamic acid residues, that interacts with the peptide bonds at the ends of the LXXLL motif, thereby orienting and anchoring the peptide. The presence of a “charge clamp” is relatively uncommon in other LXXLL-binding interactions and allows for the generation of greater selectivity for the nuclear receptors. The identity of the amino acids that flank the LXXLL motif also contribute to the selectivity and affinity of the peptide. This disclosure provides compounds that disrupt the interaction between the peptide and the agonist-liganded AR based on the internal (LXXLL) factor, the interactions with the “charge clamp” residues, and the influence played by the flanking amino acids.

The fundamental component present in the co-activator peptides is an α-helical structure that presents the lipophilic leucine side chains toward the binding groove which is lined with complementary lipophilic residues.

The compounds of this disclosure act as proteomimetic agents and bind to the NRB binding site. As a result, the interaction between co-regulatory proteins and the liganded AR is inhibited because the co-regulatory protein cannot bind to the liganded AR. As a further result, cell proliferation in AR-responsive cells and cancer cells may also be inhibited. In the instance that cell proliferation in cancer cells is inhibited, cancer cell stasis or death results.

### 5. METHODS OF TREATMENT

As discussed above, the novel compounds of this disclosure are useful for inhibiting androgen and estrogen signaling both in vivo and in vitro. These compounds, or pharmaceutically acceptable salts thereof, are useful for administration in therapeutically effective amounts for inhibiting androgen signaling, estrogen signaling, and cell proliferation in a subject. Such inhibition is useful for treating cancer in a subject as described below.

A. Prostate Cancer

Novel compounds of this disclosure can interact with a prostate cancer target to inhibit cellular proliferation. Specifically, compounds of Formulae (I) and (IA) selectively disrupt androgen signaling mechanisms in prostate cancer cells, causing cancer cell stasis or death, while leaving non-cancer, androgen-responsive cells unaffected. These have submicromolar affinity (or Ki) for the AR-coactivator binding site, at least a 10:1 selectivity for specific AR-coactivators and AR versus other NR binding sites, submicromolar antiproliferative activity in AR-(+)-prostate cancer cells, at least 20:1 selectivity for prostate vs nonprostate cancer cells, oral activity in mice against prostate cancer xenografts, and no significant acute or chronic toxicity.

B. Breast Cancer

The compounds of this disclosure can also inhibit estrogen signaling and cell proliferation in breast cancer targets. These compounds exhibit sub-micromolar affinity for the estrogen receptor NRB binding site and selectivity for that site. Thus, the compounds of this disclosure are useful for treating hormone-responsive breast cancer. The compounds selectively disrupt estrogen signaling mechanisms in breast cancer cells, causing cancer cell stasis or death, while leaving non-cancer, estrogen responsive cells unaffected.

C. Formulation

This disclosure is also directed to a pharmaceutical formulation comprising at least one compound of Formula (I) or (IA), and a pharmaceutically-acceptable carrier which is suitable for administration to a subject. These pharmaceutical formulations can be used for treating a disorder such as those described supra.

Any suitable pharmaceutically acceptable carrier known in the art can be used as long as it does not affect the inhibitory activity of a compound of Formula (I) or (IA). Carriers may be used that efficiently solubilize the agents. Carriers include, but are not limited to, a solid, liquid, or a mixture of a solid and a liquid. The carriers can take the form of capsules, tablets, pills, powders, lozenges, suspensions, emulsions, or syrups. The carriers can include substances that act as flavoring agents, lubricants, solubilizers, suspending agents, binders, stabilizers, tablet disintegrating agents, and encapsulating materials. Other examples of suitable physiologically acceptable carriers are described in Remington's Pharmaceutical Sciences (21st ed. 2005), incorporated into this disclosure by reference.

Non-limiting examples of materials which can serve as pharmaceutically-acceptable carriers include: sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline, Ringer's solution, ethyl alcohol; phosphate buffer solutions; and other non-toxic compatible substances employed in pharmaceutical formulations.

The formulations can conveniently be presented in unit dosage form and can be prepared by any methods known in the art of pharmacy. The amount of compound of Formula (I) or (IA) which can be combined with a carrier material to produce a single-dosage form will vary depending upon the subject being treated, the particular mode of administration, the particular condition being treated, among others. The amount of active ingredient that can be combined with a carrier material to produce a single-dosage form will generally be that amount of the compound that produces a therapeutic effect. Generally, out of one hundred percent, this amount will range from about 1% to about 99% of active ingredient, in some instances from about 5% to about 70%, in other instances from about 10% to about 30%.

Methods of preparing these formulations or compositions include the step of bringing into association a compound disclosed in this disclosure with a carrier and, optionally, one or more accessory ingredients. In general, the formulations are prepared by uniformly and intimately bringing into association a compound of Formula (I) or (IA) with liquid carriers, or timely divided solid carriers, or both, and then, if necessary, shaping the product.

In solid dosage forms of the disclosed compounds for oral administration (e.g., capsules, tablets, pills, dragees, powders, granules, and the like), the active ingredient is mixed with one or more additional ingredients, such as sodium citrate or dicalcium phosphate, and/or any of the following: fillers or extenders, such as, but not limited to, starches, lactose, sucrose, glucose, mannitol, and/or silicic acid; binders, such as, but not limited to, carboxymethylcellulose, alginates, gelatin, polyvinyl pyrrolidone, sucrose, and/or acacia; humectants, such as, but not limited to, glycerol; disintegrating agents, such as, but not limited to, agar, calcium carbonate, potato or tapioca starch, alginic acid, certain silicates, and sodium carbonate; solution retarding agents, such as, but not limited to, paraffin; absorption accelerators, such as, but not limited to, quaternary ammonium compounds; wetting agents, such as, but not limited to, cetyl alcohol and glycerol monostearate; absorbents, such as, but not limited to, kaolin and bentonite clay; lubricants, such as, but not limited to, talc, calcium stearate, magnesium stearate, solid polyethylene glycols, sodium lauryl sulfate, and mixtures thereof; and coloring agents. In the case of capsules, tablets, and pills, the pharmaceutical compositions can also comprise buffering agents. Solid compositions of a similar type can also be employed as fillers in soft and hard-filled gelatin capsules using such excipients as lactose or milk sugars, as well as high molecular weight polyethylene glycols, and the like.

In powders, the carrier is a finely-divided solid, which is mixed with an effective amount of a finely-divided agent. Powders and sprays can contain, in addition to a compound of Formula (I) or (IA), excipients, such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Tablets for systemic oral administration can include one or more excipients as known in the art, such as, for example, calcium carbonate, sodium carbonate, sugars (e.g., lactose, sucrose, mannitol, sorbitol), celluloses (e.g., methyl cellulose, sodium carboxymethyl cellulose), gums (e.g., arabic, tragacanth), together with one or more disintegrating agents (e.g., maize, starch, or alginic acid, binding agents, such as, for example, gelatin, collagen, or acacia), lubricating agents (e.g., magnesium stearate, stearic acid, or talc), inert diluents, preservatives, disintegrants (e.g., sodium starch glycolate), surface-active and/or dispersing agent. A tablet can be made by compression or molding, optionally with one or more accessory ingredients.

In solutions, suspensions, emulsions or syrups, an effective amount of a disclosed compound is dissolved or suspended in a carrier, such as sterile water or an organic solvent, such as aqueous propylene glycol. Other compositions can be made by dispersing the agent in an aqueous starch or sodium carboxymethyl cellulose solution or a suitable oil known to the art. The liquid dosage forms can contain inert diluents commonly used in the art, such as, for example, water or other solvents, solubilizing agents and emulsifiers, such as, but not limited to, ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan, and mixtures thereof.

Besides inert diluents, the oral compositions can also include adjuvants, such as wetting agents, emulsifying and suspending agents, sweetening, flavoring, coloring, perfuming, and preservative agents.

Suspensions can contain, in addition to the active compound, suspending agents as, for example, ethoxylated isostearyl alcohols, polyoxyethylene sorbitol and sorbitan esters, microcrystalline cellulose, aluminum metahydroxide, bentonite, agar and tragacanth, and mixtures thereof.

Formulations of the pharmaceutical compositions for rectal or vaginal administration can be presented as a suppository, which can be prepared by mixing one or more compounds of this disclosure with one or more suitable non-irritating excipients or carriers comprising, for example, cocoa butter, polyethylene glycol, a suppository wax or a salicylate, and which is solid at RT but liquid at body temperature and, thus, will melt in the rectum or vaginal cavity and release the agents. Formulations suitable for vaginal administration also include, but are not limited to, pessaries, tampons, creams, gels, pastes, foams, or spray formulations containing such carriers as are known in the art to be appropriate.

Dosage forms for the topical or transdermal administration of a compound of this disclosure include, but are not limited to, powders, sprays, ointments, pastes, creams, lotions, gels, solutions, patches, and inhalants. The active compound can be mixed under sterile conditions with a pharmaceutically-acceptable carrier, and with any preservatives, buffers, or propellants.

Ointments, pastes, creams, and gels can contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of a compound of Formula (I) or (IA) to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the compound in a polymer matrix or gel.

The compounds of Formula (I) or (IA) are administered in a therapeutically effective amount to a patient in need of such treatment. Such an amount is effective in treating a disorder of the patient. This amount can vary, depending on the activity of the agent utilized, the nature of the disorder, and the health of the patient. A skilled practitioner will appreciate that the therapeutically-effective amount of a compound of Formula (I) or (IA) can be lowered or increased by fine-tuning and/or by administering more than one compound of Formula (I) or (IA), or by administering a compound of Formula (I) together with a second agent (e.g., antibiotics, antifungals, antivirals, NSAIDS, DMARDS, steroids, etc.). Therapeutically-effective amounts can be easily determined, for example, empirically by starting at relatively low amounts and by step-wise increments with concurrent evaluation of beneficial effect (e.g., reduction in symptoms). The actual effective amount will be established by dose/response assays using methods standard in the art (Johnson et al., Diabetes (1993) 42:1179). As is known to those in the art, the effective amount will depend on bioavailability, bioactivity, and biodegradability of the compound of Formula (I) or (IA).

A therapeutically-effective amount is an amount that is capable of reducing a symptom of a disorder in a subject. Accordingly, the amount will vary with the subject being treated. Administration of the compound of Formula (I) or (IA) can be hourly, daily, weekly, monthly, yearly, or a single event. For example, the effective amount of the compound can comprise from about 1 μg/kg body weight to about 100 mg/kg body weight. In one embodiment, the effective amount of the compound comprises from about 1 μg/kg body weight to about 50 mg/kg body weight. In a further embodiment, the effective amount of the compound comprises from about 10 μg/kg body weight to about 10 mg/kg body weight. When one or more compounds of Formula (I) or (IA) or agents are combined with a carrier, they can be present in an amount of about 1 weight percent to about 99 weight percent, the remainder being composed of the pharmaceutically-acceptable carrier.

D. Administration

Methods of administration of the therapeutic formulations comprising the compounds of Formula (I) or (IA) can be by any of a number of methods known in the art. These methods include, but are not limited to, local or systemic administration. Exemplary routes of administration include, but are not limited to, oral, parenteral, transdermal, intradermal, intramuscular, intraperitoneal, intravenous, subcutaneous, intranasal (e.g., nebulizer, inhaler, aerosol dispenser), colorectal, rectal, intravaginal, and any combinations thereof. In addition, it may be desirable to introduce pharmaceutical compositions of the disclosed compounds into the central nervous system by any suitable route, including intraventricular and intrathecal injection. Intraventricular injection can be facilitated by an intraventricular catheter, for example, attached to a reservoir, such as an Ommaya reservoir. Methods of introduction can be provided by rechargeable or biodegradable devices, e.g., depots. Furthermore, administration can occur by coating a device, implant, stent, or prosthetic. The compounds of Formula (I) or (IA) can also be used to coat catheters in any situation where catheters are inserted in the body.

The therapeutic formulations containing a compound of Formula (I) or (IA) can also be administered as part of a combinatorial therapy with other agents. Combination therapy refers to any form of administration combining two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either simultaneously or sequentially. Thus, an individual who receives such treatment can have a combined (conjoint) effect of different therapeutic compounds.

In the case of cancer, the subject compounds can be administered in combination with one or more anti-angiogenic factors, chemotherapeutics, or as an adjuvant to radiotherapy. It is further envisioned that the administration of the subject compounds will serve as part of a cancer treatment regimen, which may combine many different cancer therapeutic agents.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Testing of Compounds

Some compounds of this disclosure were tested in two of the assays used to evaluate coactivator binding inhibitor activity and a radiometric binding assay to determine the potential of the compounds to act as ligands. The compounds tested are shown in Table 1.

TABLE 1

| | | |
|---|---|---|
| 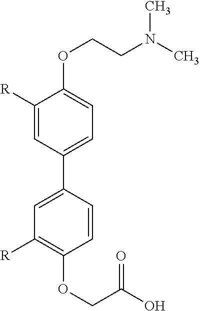 | PW-83 PW-80 AW-87 AW-82 PW-81 | R = H R = isopropyl R = sec-butyl R = tert-butyl R = benzyl |
| 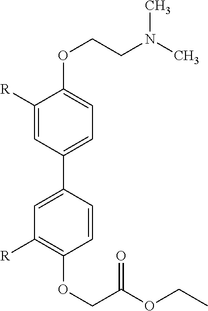 | PW-82 PW-76 AW-79 AW-72 PW-78 | R = H R = isopropyl R = sec-butyl R = tert-butyl R = benzyl |
| 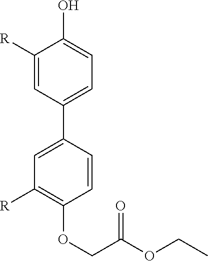 | AW-84 PW-74 AW-78 AW-73 PW-77 | R = H R = isopropyl R = sec-butyl R = tert-butyl R = benzyl |

### 1. Coactivator Binding Inhibition Assays: In Vitro (TR-FRET) and in Cells (Reporter Gene)

The protocols for both the time-resolved fluorescence resonance energy transfer (TR-FRET) assay and the cell-based reporter gene assay can be found in Parent, et al. (2008) J. Med. Chem. 51(20):6512-30. A 15-mer SRC1-Box II peptide and a published guanylhydrazone CBI (Lafrate et al., (2008) Bioorg. Med. Chem. 2008:16(23)) were used as positive controls in the TR-FRET and reporter gene assays, respectively.

A. TR-FRET CBI Assay

Purified ERα-417, labeled site-specifically with maleimide-biotin (Quanta BioDesign) and SRC-3-NRD, labeled nonspecifically through cysteine residues with iodoacetamide-fluorescein (Invitrogen), were used in the TR-FRET assays. A 5 μL volume of a stock solution mix of ERα-417 (8 nM), estradiol (4 μM), and LanthaScreen streptavidin-terbium (Invitrogen) (2 nM) in TR-FRET buffer (20 mM Tris, pH 7.5, 0.01% NP40, 50 mM NaCl) were placed in separate wells of a black 96-well Molecular Devices HE high efficiency microplate (Molecular Devices, Inc., Sunnyvale, Calif.). In a second 96-well Nunc polypropylene plate (Nalge Nunc International, Rochester, N.Y.), a 0.02 M solution of each coactivator binding inhibitor was serially diluted in a 1:10 fashion into DMF. Each concentration of coactivator binding inhibitor or vehicle was then diluted 1:10 into TR-FRET buffer, and 10 μL of this solution was added to the stock Elba solution in the 96-well plate. After a 20 min incubation, 5 μL of 200 nM fluorescein-SRC-3-NRD was added to each well. This mixture was allowed to incubate for 1 h at RT in the dark. TR-FRET was measured using an excitation filter at 340/10 nm and emission filters for terbium and fluorescein at 495/20 and 520/25 nm, respectively. The final concentrations of the reagents were as follows: ERα-417 (2 nM), streptavidin-terbium (0.5 nM), estradiol (1 μM), coactivator binding inhibitor (0-1 mM), SRC-3-NRD (50 nM).

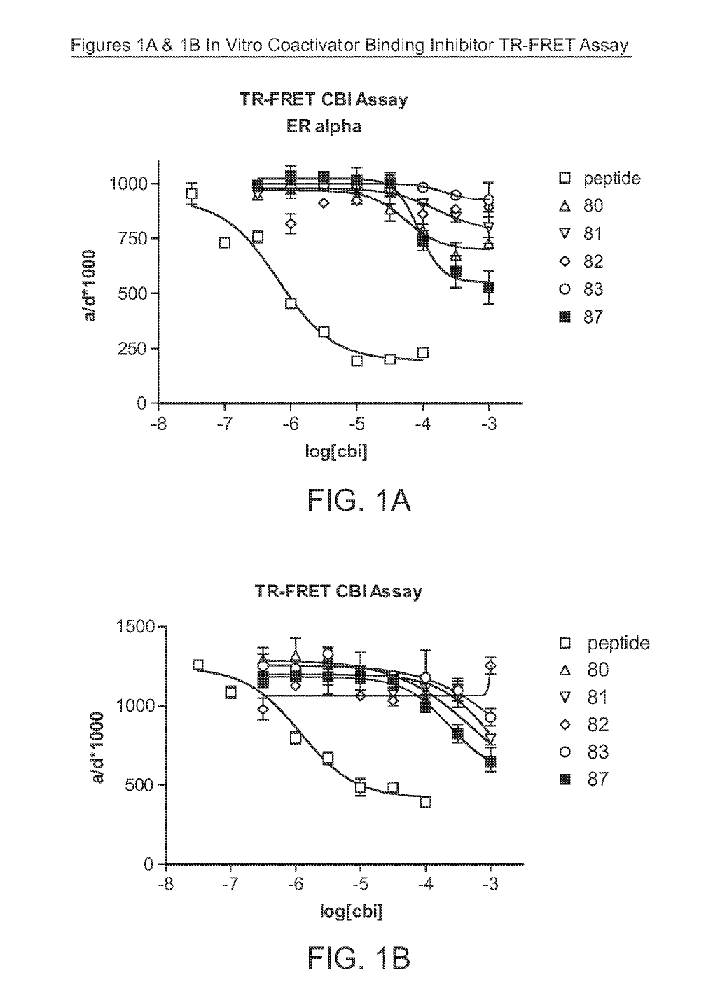

This assay was run in duplicate and repeated to confirm results. The results are shown in FIGS. 1A and 1b and show the displacement of the SRC3. The peptide control has an IC₅₀ of about 1 uM.

B. Luciferase Reporter Gene Assay

Human endometrial cancer (HEC-1) cells were maintained in culture and transfected in 24 well plates. HBSS (50 μL/well), Holo-transferrin (Sigma T1408) (20 μL/well), and lipofectin (Invitrogen no. 18292-011) (5 μL/well) were incubated together at RT for 5 min. A DNA mixture containing 200 ng of pCMVβ-galactosidase as an internal control, 500 ng of the estrogen responsive reporter gene plasmid 2ERE Luc, and 100 ng of full-length ER alpha expression vector with 75 μL of HBSS per well was added to the first mixture and allowed to incubate for 20 min at RT. After changing the cell media to Opti-MEM (350 μL/well), 150 μL of the transfection mixture was added to each well. The cells were incubated at 37° C. in a 5% CO₂ containing incubator for 6 h before the medium was replaced with fresh medium containing 5% charcoal-dextran-treated calf serum and the desired concentrations of ligands. Luciferase reporter gene activity was assayed 24 h after ligand addition.

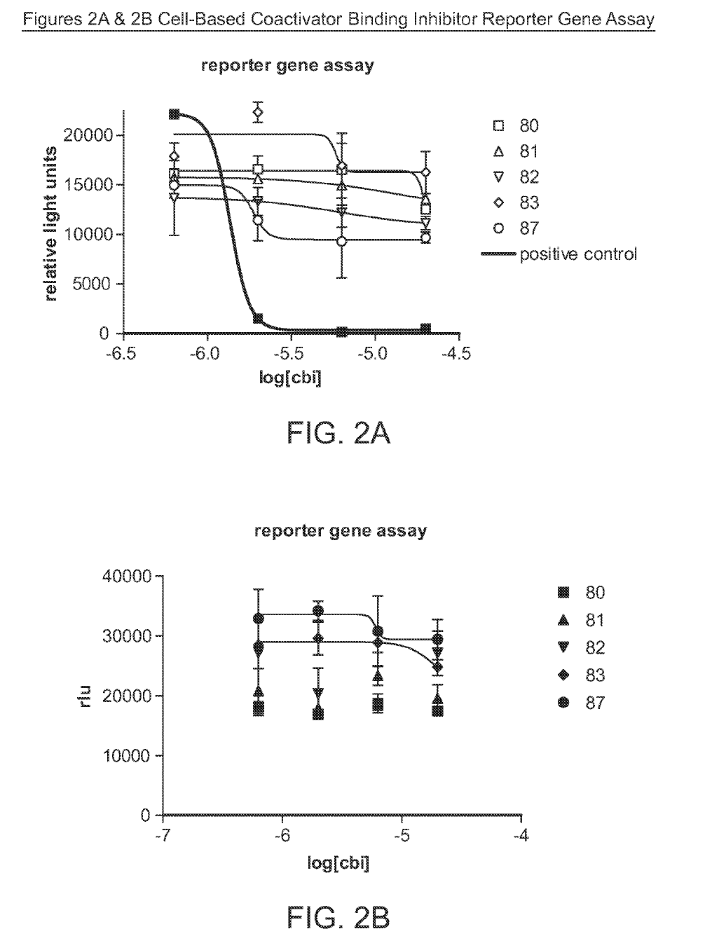

In the initial screen, compounds were assayed in a dose-response format at concentrations ranging from 20 μM to 0.6 μM; their inhibitory potential was determined by performing the assay in the presence of 10-9 M estradiol (E2). Upon validation of antagonistic activity, mechanism of action was examined by repeating the compound titration in the presence of both 10-7 and 10-9 M E2 with an expectation that changing the concentration of E2 100-fold would not change the IC₅₀ of true coactivator binding inhibitors. These assays was also repeated to confirm results. The positive control is a guanyl hydrazone. The results are shown in FIGS. 2A and 2B.

A summary of CBI activity in the TR-FRET and Reporter Gene assays is shown in Table 2.

TABLE 2

| | | TR-FRET | | Reporter Gene |
|---|---|---|---|---|
| | TR-FRET | Repeat | Reporter Gene | IC₅₀ (μM) - |
| Compound | IC₅₀ (μM) | (μM) | IC₅₀ (μM) | repeat |
| Control | 0.7 | 1.1 | 2 | — |
| 80 | 55* | 583* | 21* | — |
| 81 | 163* | 1095* | 13* | — |
| 82 | — | — | 6.0* | — |
| 83 | 205* | 2046* | 5.8* | 18* |
| 87 | 87 | 227 | 1.8* | 6* |

*very small dynamic range compared to positive control peptide or guanylhydrazone CBI

C. Estrogen Receptor-Relative Binding Affinity Assay

Relative binding affinity assays were run to determine whether the compounds of this disclosure are traditional ligands that compete with estradiol. Relative binding affinities were determined using a modification of a competitive radiometric binding assay, using 2 nM [3H]estradiol as tracer ([2,4,6,7-3H]estra-1,3,510-triene-3,173-diol, 84-85 Ci/mmol, Amersham/GE Health Care, Piscataway, N.J.); purified full-length human ERα and ERβ were purchased from PanVera/Invitrogen (Carlsbad, Calif.). Incubations were conducted for 18-24 h at 0° C. Hydroxyapatite (BioRad, Hercules, Calif.) was used to absorb the receptor-ligand complexes, and free ligand was washed away. The binding affinities are expressed as relative binding affinity (RBA) values with the RBA of estradiol set to 100%. Estradiol binds to ERα with a Kd of 0.2 nM and to ERβ with a Kd of 0.5 nM.

The results of the radiometric binding assay of CBI compounds are shown in Table 3.

TABLE 3

| Compound | ERα RBA | ERβ RBA |
|---|---|---|
| PW-I-80 | <0.001 | <0.001 |
| PW-I-81 | <0.001 | ~0.001 |
| PW-I-83-3 | <0.001 | <0.001 |
| AW-III-82-56 | 0.055 | 0.018 |
| | 0.038 | 0.017 |
| | 0.049 ± 0.016 | 0.018 ± 0.001 |
| AW-III-87-68 | ~0.001 | ~0.001 |

These findings show that the compounds will not act as traditional ligand antagonists.

### 2. Affinity Assays

The Mammalian Hybrid Assay used a similar procedure as described in Chang et al., Molec. & Cellul. Biol., (1999) 19:12 8226-8239 except the compounds shown in Table 1, supra, were used instead of phage-derived peptides.

A. Cell Culture and Transient Transfection

Human cervical cancer (HeLa) and hepatoma (HepG2) cells were cultured in minimum essential medium (Life Technologies, Inc.) supplemented with 10% fetal bovine serum (HyClone), 0.1 mM nonessential amino acids, and 1 mM sodium pyruvate (Life Technologies, Inc.) and maintained in a humidified 37° C. incubator with 5% CO2. For transient transfections, cells were split into 24-well plates 24 h before transfection. Lipofectin (Life Technologies, Inc.)-mediated transfection has been described in detail previously (27). A DNA-Lipofectin mixture containing a total of 3,000 ng of plasmid in each of triplicate samples was incubated with cells for 3 to 5 h, and transfection was stopped by replacing the transfection mix with fresh medium (minimal essential medium without phenol red) containing 10% charcoal-stripped serum. Receptor ligands were added to the cells 14 to 16 h before the assay. Luciferase and (3-galactosidase activities were measured. In mammalian two-hybrid assays, for a typical triplicate of transfection, 2,000 ng of 5×Gal4Luc3 reporter plasmid, 400 ng of receptor-VP16 fusion, 400 ng of pM (Gal4 DBD)-peptide fusion constructs, and 200 ng of normalization plasmid pCMVβgal were used. For ER transcription disruption assays, 1,600 ng of 3 XERE-TATA-Luc reporter, 200 ng of pCMVβgal, 400 ng of either pRST7ERα, pRST7ERβ, or other receptor mutant constructs, and 0 to 800 ng of pM-peptide fusion plasmids were used. The parent pM vector (Gal4DBD without peptide fusion) was used in these experiments to balance the amount of input DNA in transfections. All transfections were performed at least three times.

B. Affinity Selection of ERα-Binding Sequences

Baculovirus-expressed full-length ERα was provided by PanVera Corp. (Madison, Wis.). Approximately 0.25 μg (4 pmol) of ERα was diluted in 100 μl of NaHCO₃ (pH 8.5) plus 10-6 M 17β-estradiol, applied to a single well in a 96-well Immulon 4 plate (Dynex Technologies, Inc.), and incubated at RT for 3 h. An equal amount of BSA was added to the adjacent well as a control target. The wells were blocked with 150 μl of 0.1% BSA in NaHCO₃ for an additional 1 h at RT and washed five times with PBST (137 mM NaCl, 2.7 mM KCl, 4.3 mM Na₂HPO₄, 1.4 mM KH₂PO₄ [pH 7.3], 0.1% Tween 20) to remove excess protein. Then 25 μl of the stock solution containing the compounds to be tested was diluted in 125 μl of PBST with 10-6 M 17β-estradiol and 0.1% BSA was added to the wells, and the plate was sealed and incubated for 8 h at RT. Nonbinding compounds were removed by washing the wells five times with PBST.

C. Assay Results

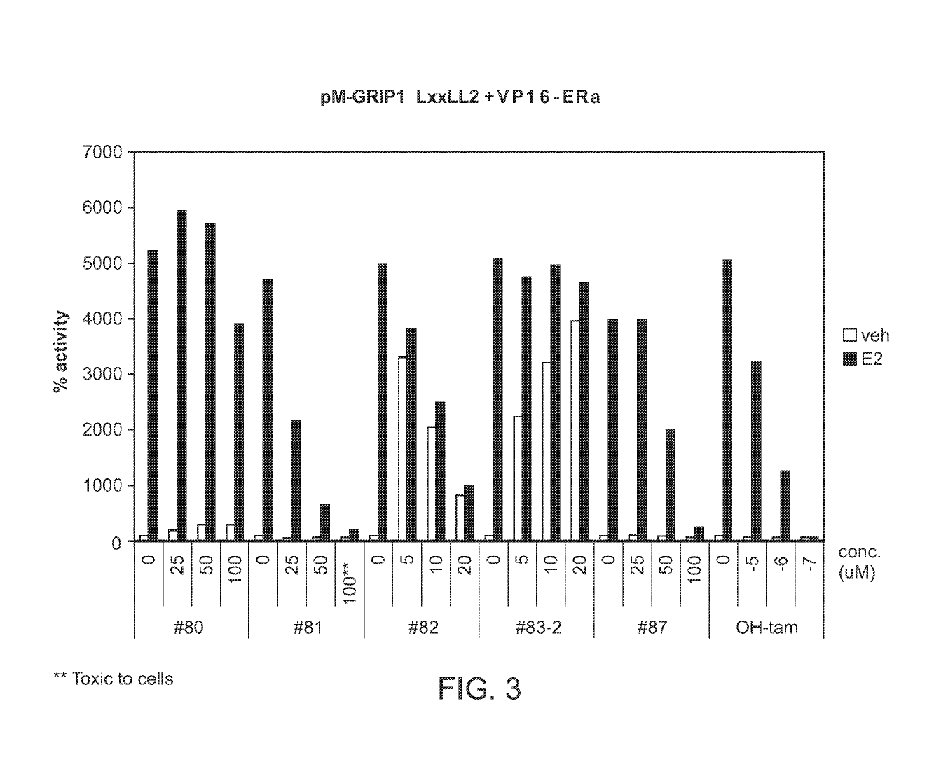

The mammalian two hybrid assay assessed whether the compounds disrupt the interaction between ERα and an LXXLL peptide derived from an ER coactivator (GRIP1). OH-tam is an ERα antagonist and was used as a positive control. The results, which are shown in FIG. 3, show that the compounds interrupt the interaction.

A second assay with another group of compounds was performed to determine the proper dose for use in cell culture. Compounds were used at concentrations between 5-40 μM. The mammalian two hybrid assay was used to examine the interaction between ERα and the LXXLL2 peptide from the coactivator GRIP1. #82 and OH-tam were used as controls.

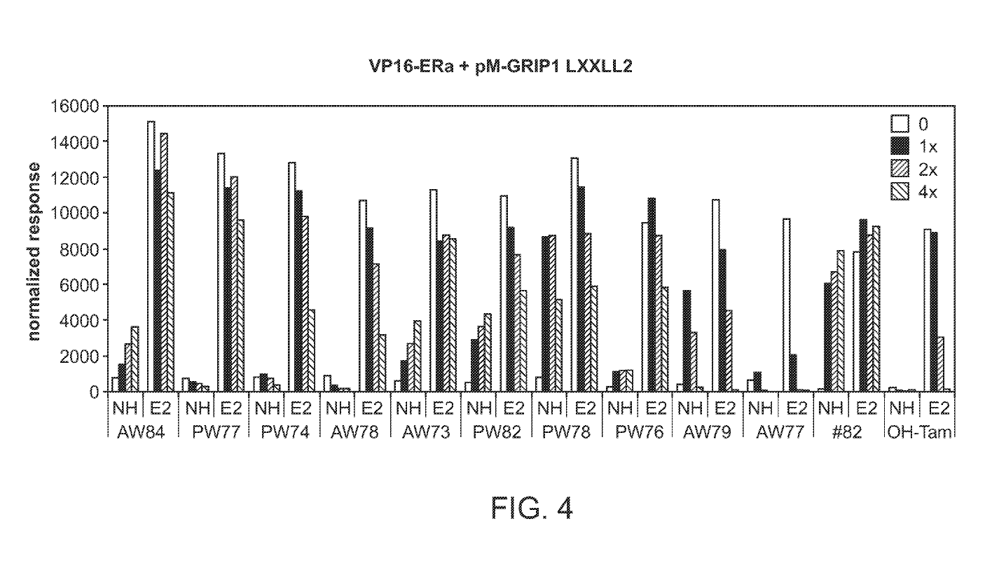

The results are shown in FIG. 4. PW74 inhibits at about 40 μM and no significant toxicity was seen at this dose. AW78 was also effective. AW84, AW73, PW82, PW78, PW76, and AW79 all induced LXXLL-ER interaction. AW 79 was toxic at a high dose. AW77 was very toxic to cells at the high concentrations tested.

D. Quantitative-PCR Analysis of ERα Target Genes in MCF7 (ERα Positive) Breast Cancer Cells

MCF7 cells were pre-treated with 20 μM of each compound tested for 2 h and then either vehicle or estradiol was added for 14 h. RNA was harvested and quantitative-PCR was used to determine the effects of these compounds on the expression of ERα target genes. SDF1, PR, and PS2 are known ERα target genes. IDH3A is not an ERα target gene and was used as a negative control to demonstrate the selectivity of these compounds.

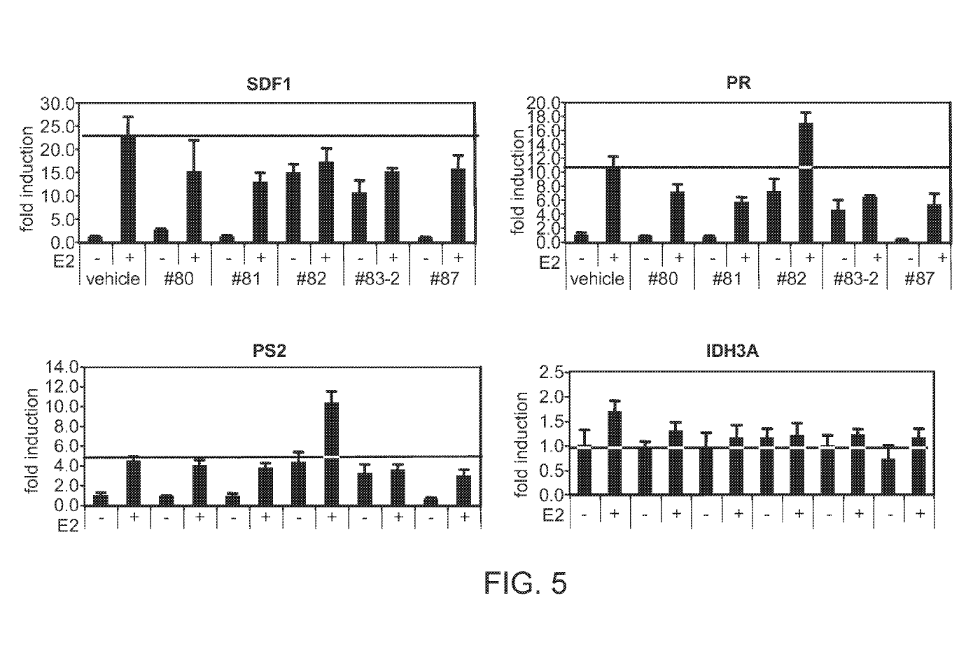

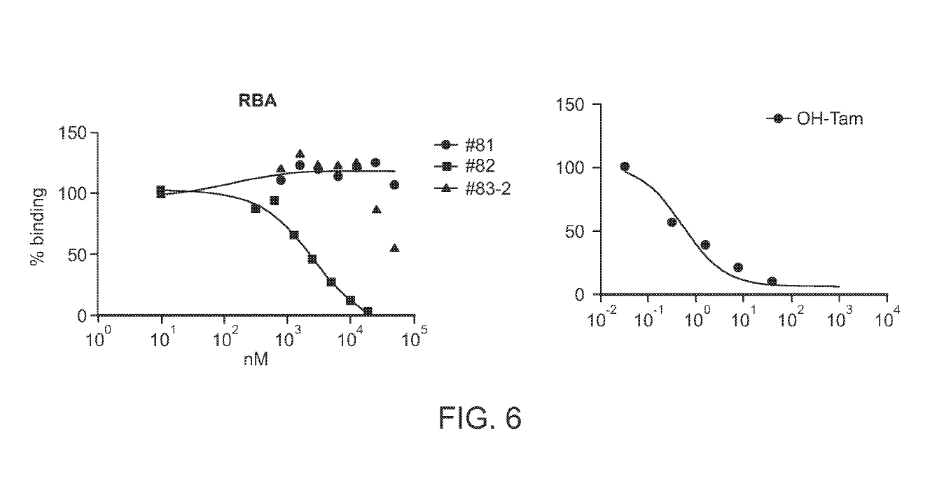

At the concentration of 20 μM, the compounds showed inhibition of the expression the ERα target genes as shown in FIG. 5.

E. RBA Assay

The RBA assay was performed to examine the ability of the compounds to compete for the binding of 1 nM of 3H-estradiol to the endogenous ERα in MCF7 cells. The results are shown in FIG. 5. Compound #81 did not compete, while compounds #82 and #83-2 both competed estradiol from binding.

### Example 2

### Evaluation of Compounds as Selective Inhibitors of AR-Coactivator and ER-Coactivator Binding

The compounds of this disclosure are analyzed for selectivity for AR and/or ER. For controls, peptide sequences are obtained that are selective for each of the nuclear receptors tested. Negative controls consist of vehicle and a random sequence helical peptide. The results are graphed, and Kᵢ values determined from the curves for each compound and peptide tested. Compounds with Kᵢ values in the 0.05-1.0 μM range and AR and/or ER selectivity in the 10-20-fold range are expected.

### Example 3

### Evaluation of Compounds in AR-Responsive and AR-Independent Cells as Inhibitors of Cell Proliferation

Compounds of this disclosure that are selective inhibitors of AR-coactivator binding are evaluated initially in prostate cancer cell lines (PC-3 and DU-145) as inhibitors of cell proliferation. These two cell lines are standard lines for evaluating AR-mediated effects, the former being AR-positive and the latter being AR-independent. Both lines are examined in the presence or absence of androgens, anti-androgens and vehicle to determine the effects of the compounds of this disclosure and whether the responses are mediated through co-activator or co-repressor binding sites. The responses of the compounds on other non-prostate cancer cell lines, such as normal fibroblasts and breast cancer (MCF-7) cells, are used to illustrate their selectivity for prostate cancer. In addition to determining the anti-proliferative response, the effect of the compounds on cellular proteins using gel electrophoresis is also examined. Expected results show that a compound selectively inhibits proliferation of prostate cancer cells with intracellular responses associated with AR.

### Example 4

### Evaluation of Selected Compounds in Tumor-Bearing SCID Mice

Prior to evaluating the anti-tumor response of candidate compound, safety in mice is determined using escalating i.p. and p.o. dosing schedules. The cellular assays provide an estimated concentration needed to effect the requisite cell proliferation response. This gives a targeted dose range in the tumor-bearing SCID mouse study. PC-3 and DU-145 xenografts are used in male nude mice to evaluate the effectiveness and selectivity against AR-responsive tumors. These are standard animal models for testing AR-directed therapies and the results as compared to animals receiving anti-hormonal, doxorubicin, or sham therapy. Success in this study is defined as no overt toxicity in non-tumor bearing animals, a reduction in tumor growth compared to control animals, and selectivity for AR-positive versus AR-independent tumors.

### Example 5

### Evaluation of Compounds in ER-Responsive and ER-Independent Cells as Inhibitors of Cell Proliferation

Compounds are evaluated initially in breast cancer cell lines (MCF-7 and MDA-MB-231) as inhibitors of cell proliferation. These two cell lines are standard lines for evaluating ER-mediated effects, the former being ER-positive and the latter being ER-independent. Both lines are examined in the presence or absence of estrogens, anti-estrogens and vehicle to determine the effects of the compounds and whether the responses are mediated through co-activator or co-repressor binding sites. The responses of the compounds on other non-breast cancer cell lines, such as normal fibroblasts and prostate cancer (PC-3) cells are examined to illustrate their selectivity for breast cancer. In addition to determining the anti-proliferative response, the effect of the compounds on cellular proteins using gel electrophoresis is evaluated. Expected results show that a compound that selectively inhibits proliferation of breast cancer cells at submicromolar concentrations with intracellular responses associated with ER.

### Example 6

### Evaluation of Selected Compounds in Tumor-Bearing SCID Mice

Prior to evaluating the anti-tumor response, safety in mice is determined using escalating i.p. and p.o. dosing schedules. The cellular assays provide an estimated concentration needed to generate the requisite cell proliferation response. This gives a targeted dose range in our tumor-bearing SCID mouse study. MCF-7 and MDA-MB-231 xenografts are used in female nude mice to evaluate the effectiveness and selectivity against ER-responsive tumors. These are standard animal models for testing ER-directed therapies and the results will be compared to animals receiving anti-hormonal, doxorubicin, or sham therapy. Success in this study is defined as no overt toxicity in non-tumor bearing animals, a reduction in tumor growth compared to control animals, and selectivity for ER-positive vs. ER-independent tumors.

### Example 7

### Synthesis of Some Exemplary Compounds

All commercially available reagents were purchased from Sigma-Aldrich and used without further purification. Solvents were distilled and reactions requiring inert conditions were performed under N₂ or argon. Column chromatography was performed using silica gel unless otherwise indicated. Thin layer chromatography was used to monitor reactions using phosphomolybdic acid and/or iodine vapor. ¹H- and 13C-NMR spectra were recorded on a Bruker 500 mHz spectrometer using CDCL₃ or d₆-acetone as the solvent. High resolution mass spectral data were obtained at the University of Massachusetts Mass Spectrometry Facility which is supported, in part, by the National Science Foundation.

### Synthesis of Para-Brominated 2-Alkylphenols

### 1. Preparation of 2-benzyl-4-bromophenol (3e)

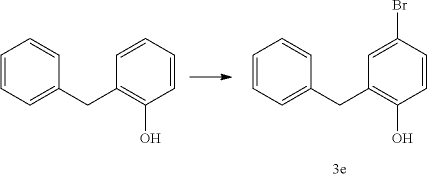

2-benzylphenol (4.61 g, 25 mmol) was dissolved in chloroform (200 ml). Tetrabutyl ammonium tribromide (14.47 g, 30 mmol) was added and the solution was stirred for 3 h at RT. The solvent was evaporated and the crude product was partitioned between ether and water. The ether layer was washed with 1 N HCl (2×) and brine (2×). The organic layer was extracted and dried over magnesium sulfate. Solvent was evaporated and the crude product was chromatographed over silica gel (Hexane/EtOAc, 80:20). 6.2 g (94%) of desired product was isolated as a clear yellow oil.

### 2. 2-isopropyl-4-bromophenol (3b)

The procedure for 3e was repeated. Product was isolated in 88% yield as a yellow oil.

### 3. 2-sec-butyl-4-bromophenol (3c)

The procedure for 3e was repeated. The product was isolated in 85% yield as a yellow oil.

The compound was confirmed as follows: ¹H NMR (500 MHz, CDCl₃-d): δ 7.24 (d, J=2.5 Hz, 1H), 7.16 (dd, =8 Hz, 2.5 Hz, 1H, 6.65 (d, J=8 Hz, 1H), 4.82 (br s, 1H), 2.93 (m, 1H), 1.62 (m, 2H), 1.22 (d, J=6.5 Hz, 3H), 0.877 (t, J=7 Hz, 3H) ppm.

### 4. 2-tert-butyl-4-bromophenol (3d)

The procedure for 3e was repeated. The product was isolated in 82% yield as a yellow oil.

The compound was confirmed as follows: ¹H NMR (500 MHz, CDCl₃-d): δ 7.36 (d, J=2.5 Hz, 1H), 7.17 (dd, J=8.5 Hz, 2.5 Hz, 1H), 6.57 (d, J=8 Hz, H), 5.010 (br s, 1H), 1.396 (s, 9H) ppm.

### Example 8

### Synthesis of Para-Hydroxyphenylboronate Esters

### 1. Preparation of 2-benzyl-4-(4,4,5,5-tetramethyl-1,3,2-dioxaborolan-2-yl)phenol (4e)

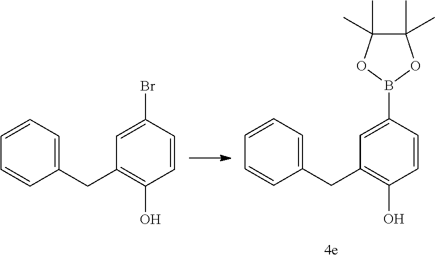

2-benzyl-4-bromophenol (3e) (0.79 g, 3.0 mmol), PdCl₂(dppf) (0.15 g, 6 mol %), and potassium acetate (0.59 g, 6.0 mmol) were added to the reaction vessel which was vacuumed and flushed with argon. Anhydrous dioxane (20 ml) was added via syringe. The reaction was heated to 80° C. and stirred for 30 min. Bis(pinacolato)diboron (0.84 g, 3.3 mmol), dissolved in 5 ml dioxane, was added and the reaction was kept under inert atmosphere, stirred, and heated at 80° C. overnight. The crude mixture was filtered through activated carbon, and Celite and solvent was evaporated to dryness. The crude mixture was eluted with ethyl acetate, washed with water (×2), brine (×2) and dried over magnesium sulfate. The product was chromatographed over silica gel (Hexane/EtOAc, 80:20) to afford 0.60 g (64.5%) of the desired product as a white solid (m.p. 119-121° C.).

### 2. 2-isopropyl-4-(4,4,5,5-tetramethyl-1,3,2-dioxaborolan-2-yl)phenol (4b)

The procedure for 4e was repeated. Product was isolated in 95% yield as a white solid (m.p. 148-150° C.).

### 3. 2-sec-butyl-4-(4,4,5,5-tetramethyl-1,3,2-dioxaborolan-2-yl)phenol (4c)

The procedure for 4e was repeated. Product was isolated in 58% yield as a white solid.

The compound was confirmed as follows: ¹H NMR (500 MHz, CDCl₃-d): δ 7.61 (d, J=1.5 Hz, 1H), 7.55 (dd, J=7.5 Hz, 1.5 Hz, 1H), 6.74 (d, J=7.5 Hz, 1H), 4.93 (br s, 1H), 2.95 (m, 1H), 1.67 (m, 2H), 1.34 (s, 12H), 1.27 (d, J=6.5 Hz, 3H), 0.88 (t, J=7 Hz, 3H) ppm.

### 4. 2-tert-butyl-4-(4,4,5,5-tetramethyl-1,3,2-dioxaborolan-2-yl)phenol (4d)

The procedure for 4e was repeated. Product was isolated in 61% yield as a white solid.

The compound was confirmed as follows: ¹H NMR (500 MHz, CDCl₃-d): δ 7.72 (d, J=1.5 Hz, 1H), 7.55 (dd, J=7.5 Hz, 1.5 Hz, 1H), 6.66 (d, J=8 Hz, 1H), 4.96 (br s, 1H), 1.43 (s, 9H), 1.33 (s, 12H) ppm.

### Example 9

### Synthesis of Ethyl Phenoxyacetate Bromides

### 1. Preparation of ethyl 2-(2-benzyl-4-bromophenoxy)acetate (5e)

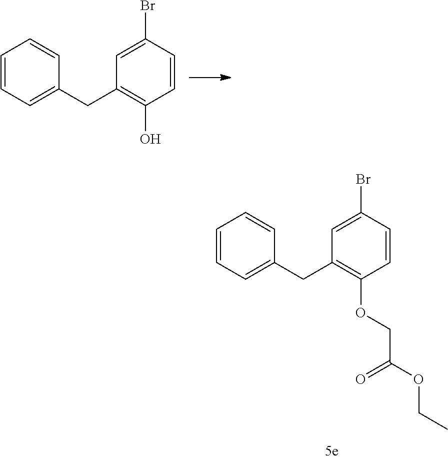

2-Benzyl-4-bromophenol (3e) (2.10 g, 8 mmol) was dissolved in dry THF (75 ml). Sodium hydride, 60% in oil, (0.45 g, 11.2 mmol) was added and the solution was stirred at RT for 30 min. 4-bromoethyl acetate (1.87 g, 11.2 mmol) was then added slowly and the solution was stirred for 2 h at RT. The reaction mixture (thick white solid) was diluted with ethyl acetate and washed with water (2×) and brine (2×). After drying over magnesium sulfate, the product was chromatographed over silica gel (Hexane/EtOAc, 90:10) to afford 2.57 g (92%) of the desired product as a crystalline solid (m.p. 67-70° C.).

### 2. Ethyl 2-(2-isopropyl-4-bromophenoxy)acetate (5b)

The procedure for 5e was repeated. Product was isolated in 78% yield as a yellow oil.

### 3. Ethyl 2-(2-sec-butyl-4-bromophenoxy)acetate (5c)

The procedure for 5e was repeated. Product was isolated in 91% yield as a pale yellow oil.

The compound was confirmed as follows: ¹H NMR (500 MHz, CDCl₃-d): δ 7.28 (d, J=2.5 Hz, 1H), 7.22 (dd, J=9 Hz, 2.5 Hz, 1H), 6.60 (d, J=9 Hz, 1H), 4.60 (s, 2H), 4.26 (q, J=7 Hz, 2H), 3.16 (m, 1H), 1.60 (m, 2H), 1.29 (t, J=7.5 Hz, 3H), 1.21 (d, J=7 Hz, 3H), 0.86 (t, J=7.5 Hz, 3H) ppm.

### 4. Ethyl 2-(2-tert-butyl-4-bromophenoxy)acetate (5d)

The procedure for 5e was repeated. Product was isolated in 82% yield as a pale yellow oil.

The compound was confirmed as follows: ¹H NMR (500 MHz, CDCl₃-d): δ 7.41 (d, J=2.5 Hz, 1H), 7.27 (dd, J=9 Hz, 2.5 Hz, 1H), 6.62 (d, J=8 Hz, 1H), 4.63 (s, 2H), 4.29 (q, J=7 Hz, 2H), 1.42 (s, 9H), 1.32 (t, J=7 Hz, 3H) ppm.

### Example 10

### Synthesis of Biphenyl Phenols

### 1. Ethyl 2-(1,1′-biphenyl-3,3′-dibenzyl-4′-ol-4-oxy)acetate (6e)

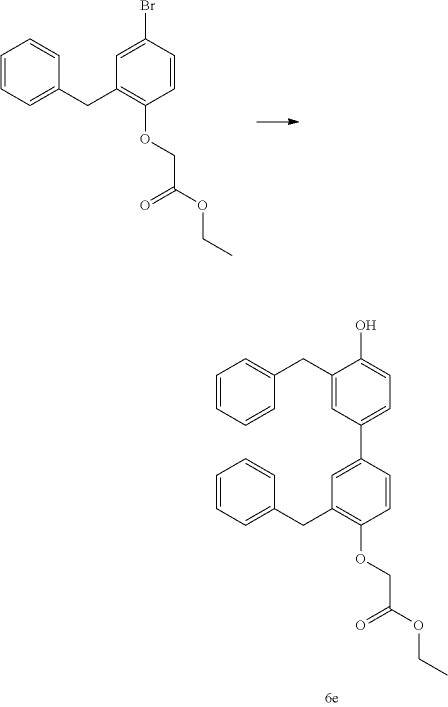

Ethyl 2-(2-benzyl-4-bromophenoxy)acetate (5e) (0.210 g, 0.60 mmol), PdCl₂(PPh₃)₂ (0.026 g, 6 mol %), PPh₃ (0.010 g, 6 mol %), and 2M aqueous sodium carbonate (0.8 ml, 1.2 mmol) were added to the reaction vessel which was vacuumed and flushed with argon. THF/water (7 ml 2 ml) was degassed with argon and added via syringe. The reaction was refluxed and stirred for 30 min. 2-Benzyl-4-(4,4,5,5-tetramethyl-1,3,2-dioxaborolan-2-yl)phenol (4e) (0.224 g, 0.72 mmol), dissolved in 3 ml dioxane, was then added and the reaction was kept under inert atmosphere, stirred at reflux overnight. The crude mixture was filtered through activated carbon, and Celite and solvent was evaporated to dryness. The crude mixture was eluted with ethyl acetate, washed with water (×2), brine (×2) and dried over magnesium sulfate. The product was chromatographed over silica gel (Hexane/EtOAc, 80:20) to afford 0.165 g (61%) of the desired product as a white solid (m.p. 142-144° C.).

The compound was confirmed as follows: ¹H NMR (500 MHz, CDCl₃): δ=7.31-7.16 (m, 14H), 6.81 (d, J=8.5 Hz, 1H), 6.77 (d, J=8.0 Hz, 1H), 4.61 (s, 2H), 4.27 (q, J=7.2 Hz, 2H), 4.09 (s, 2H), 4.03 (s, 2H), 1.30 (t, J=7.5 Hz, 3H) ppm.

### 2. ethyl 2-(1,1′-biphenyl-3,3′-diisopropyl-4′-ol-4-oxy)acetate (6b)

The procedure for 6e was repeated. Product was isolated in 49% yield as a white solid (m.p. 111-113° C.).

The compound was confirmed as follows: ¹H NMR (500 MHz, CDCl₃): δ=7.40 (d, J=2 Hz, 1H), 7.36 (d, J=2.5 Hz, 1H), 7.29 (dd, J=8.0, 2.5 Hz, 1H), 7.25 (dd, J=7.8, 2.3 Hz, 1H), 6.80 (d, J=6.5 Hz, 1H), 6.77 (d, J=7.0 Hz, 1H), 4.68 (s, 2H), 4.29 (q, J=7.0 Hz, 2H), 3.46 (7let, J=7.0 Hz, 1H), 3.26 (7let, J=7.2 Hz, 1H), 1.32 (d, J=6.5 Hz, 6H), 1.32 (t, J=7.0 Hz, 3H), 1.30 (d, J=8 Hz, 6H) ppm.

### 3. ethyl 2-(1,1′-biphenyl-3,3′-disec-butyl-4′-ol-4-oxy)acetate (6c)

The procedure for 6e was repeated. Product was isolated in 24% yield as a white solid.

The compound was confirmed as follows: ¹H NMR (500 MHz, CDCl₃-d): δ 7.36 (d, J=2 Hz, 1H), 7.32 (d, J=2 Hz, 1H), 7.30 (dd, J=8.5 Hz, 2.5 Hz, 1H), 6.82 (d, J=7.5 Hz, 1H), 6.78 (d, J=8 Hz, 1H), 4.73 (s, 1H), 4.67 (s, 2H), 4.29 (q, J=7 Hz, 2H), 3.23 (m, 1H), 3.01 (m, 1H), 1.73 (m, 2H), 1.65 (m, 2H), 1.31 (m, 9H), 0.93 (t, J=7.5 Hz, 3H), 0.91 (t, J=7.5 Hz, 3H) ppm.

### 4. ethyl 2-(1,1′-biphenyl-3,3′-didtert-butyl-4′-ol-4-oxy)acetate (6d)

The procedure for 6e was repeated. Product was isolated in 32% yield as a white solid.

The compound was confirmed as follows: ¹H NMR (500 MHz, CDCl₃-d): δ 7.48 (d, J=2.5 Hz, 1H), 7.44 (d, J=2.5 Hz, 1H), 7.32 (dd, J=2 Hz, 8.5 Hz, 1 H), 7.25 (dd, J=2 Hz, 8.5 Hz, 1H), 6.78 (d. J=8 Hz, 1H), 6.73 (d, J=7.5, 1H), 4.68 (s, 2H), 4.30 (q, J=7 Hz, 2H), 1.48 (s, 9H), 1.46 (s, 9H), 1.33 (t, J=7.5 Hz, 3H) ppm.

### Example 11

### Synthesis of the Dimethylamino Ethylester

### 1. Ethyl 2-(1,1′-biphenyl-3,3′-dibenzyl-4′-[2-(dimethylamino)ethoxy]-4-oxy)acetate (7e)

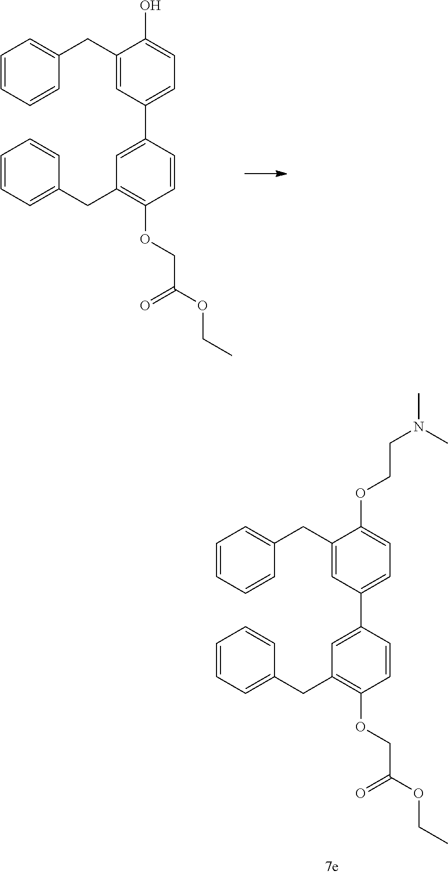

A mixture containing ethyl 2-(1,1′-biphenyl-3,3′-dibenzyl-4′-ol-4-oxy)acetate (6e) (81 mg, 0.18 mmol), 2-(dimethylamino)ethyl chloride hydrochloride (83 mg, 0.57 mmol), and potassium carbonate (132 mg, 0.95 mmol) in acetone (15 mL) was refluxed for overnight. The reaction was monitored by thin layer chromatography using C18 plates. The reaction mixture was concentrated under reduced pressure. The resultant solid was then partitioned in ethyl acetate and water, washed with brine, and dried over magnesium sulfate. The solution was concentrated under reduced pressure. The crude product was purified by column chromatography on C18 gel with ethyl acetate and hexane to yield 55 mg (60%) of the desired product as a thin colorless oil.

The compound was confirmed as follows: ¹H NMR (500 MHz, CDCl₃): δ=7.31-7.16 (m, 14H), 6.87 (d, J=9 Hz, 1H), 6.76 (d, J=8.5 Hz, 1H), 4.61 (s, 2H), 4.26 (q, J=7.5 Hz, 2H), 4.10 (t, J=5.8 Hz, 2H), 4.08 (s, 2H), 4.00 (s, 2H), 2.76 (t, J=5.5 Hz, 2H), 2.34 (s, 6H), 1.30 (t, J=7.3 Hz, 3H) ppm; ¹³C NMR (75.5 MHz, CDCl₃): δ=169.2, 155.9, 155.1, 141.2, 141.0, 134.6, 133.6, 130.8, 130.1, 129.6, 129.4, 129.3, 129.2, 129.0 128.8, 128.7, 128.3, 126.3, 126.0, 125.8, 125.6, 112.2, 112.1, 111.9, 111.7, 66.5, 66.1, 61.5, 58.3, 46.0, 45.7, 36.9, 36.4, 14.1 ppm.

### 2. ethyl 2-(1,1′-biphenyl-3,3′-diisopropyl-4′-[2-(dimethylamino) ethoxy]-4-oxy)acetate (7b)

The procedure for 7e was repeated. Product was isolated in 51% yield as a colorless oil.

The compound was confirmed as follows: ¹H NMR (500 MHz, CDCl₃): δ=7.40 (d, J=2.5 Hz, 1H), 7.37 (d, J=3.0 Hz, 1H), 7.30 (dd, J=8.5, 2.5 Hz, 1H), 7.29 (dd, J=8.5, 2.5 Hz, 1H), 6.89 (d, J=8.5 Hz, 1H), 6.76 (d, J=8.5 Hz, 1H), 4.66 (s, 2H), 4.27 (q, J=8.5 Hz, 2H), 4.13 (t, J=5.8 Hz, 2H), 3.45 (7let, J=7 Hz, 1H), 3.37 (7let, J=7 Hz, 1H), 2.81 (t, J=5.8 Hz, 2H), 2.38 (s, 6H), 1.30 (t, J=7.5 Hz, 3H), 1.29 (d, J=7 Hz, 6H), 1.26 (d, J=7 Hz, 6H) ppm; ¹³C NMR (75.5 MHz, CDCl₃): δ=169.4, 155.4, 154.5, 137.9, 137.5, 135.3, 134.1, 125.5, 125.1, 125.1, 125.0, 111.8, 111.8, 67.1, 66.1, 61.5, 58.7, 46.3, 27.4, 27.2, 23.0, 22.9, 14.4 ppm.

### 3. ethyl 2-(1,1′-biphenyl-3,3′-disec-butyl-4′-[2-(dimethylamino)ethoxy]-4-oxy)acetate (7c)

The procedure for 7c was repeated. Product was isolated in 58% yield as a colorless oil.

The compound was confirmed as follows: ¹H NMR (500 MHz, CDCl₃-d): δ 7.35 (d, J=2 Hz, 1H), 7.30 (m, 3H), 6.89 (d, J=8.5 Hz, 1H), 6.76 (d, J=8 Hz, 1H), 4.65 (s, 2H), 4.27 (q, J=7.5 Hz, 2H), 4.11 (t, J=6 Hz, 2H), 3.22 (m, 1H), 3.14 (m, 1H), 2.78 (t, J=6 Hz, 2H), 2.37 (s, 6H), 1.71 (m, 2H), 1.59 (m, 2H), 1.27 (m, 9H), 0.89 (t, J=7.5 Hz, 3H), 0.88 (t, J=7.5 Hz, 3H) ppm; ¹³C NMR (500 MHz, CDCl₃-d): δ 169.42, 155.76, 154.80, 136.86, 136.53, 135.16, 133.96, 126.14, 125.74, 125.03, 124.95, 111.92, 111.85, 67.25, 66.13, 61.44, 58.68, 46.35, 34.28, 34.00, 30.18, 30.08, 20.67, 14.40, 12.54, 12.51 ppm.

### 4. ethyl 2-(1,1′-biphenyl-3,3′-ditert-butyl-4′-[2-(dimethylamino)ethoxy]-4-oxy)acetate (7d)

The procedure for 7e was repeated. Product was isolated in 54% yield as a white solid.

The compound was confirmed as follows: ¹H NMR (500 MHz, CDCl₃-d): δ 7.48 (s, 1H), 7.45 (s, 1H), 7.33 (d, 1H), 7.31 (d, 1H), 6.93 (d, J=8.5 Hz, 1H), 6.77 (d, J=8 Hz, 1H), 4.67 (s, 2H), 4.29 (q, J=7.5 Hz, 2H), 4.15 (t, J=6.5, 2H), 2.84 (t, J=6 Hz, 2H), 2.37 (s, 6H), 1.47 (s, 9H), 1.43 (s, 9H), 1.32 (t, J=7 Hz, 3H) ppm; ¹³C NMR (500 MHz, CDCl₃-d): δ 13.15, 28.75, 28.82, 33.92, 33.98, 45.11, 57.47, 60.24, 64.40, 65.62, 110.98, 111.45, 124.16, 124.27, 124.64, 124.95, 132.53, 133.59, 137.30, 137.64, 154.59, 155.78, 167.97 ppm.

### Example 12

### Synthesis of the Carboxylic Acid and the Hydrochloride Salt

### Preparation of ethyl 2-(1,1′-biphenyl-3,3′-dibenzyl-4′-[2-(dimethylamino)ethoxy]-4-oxy)acetic acid (1e)

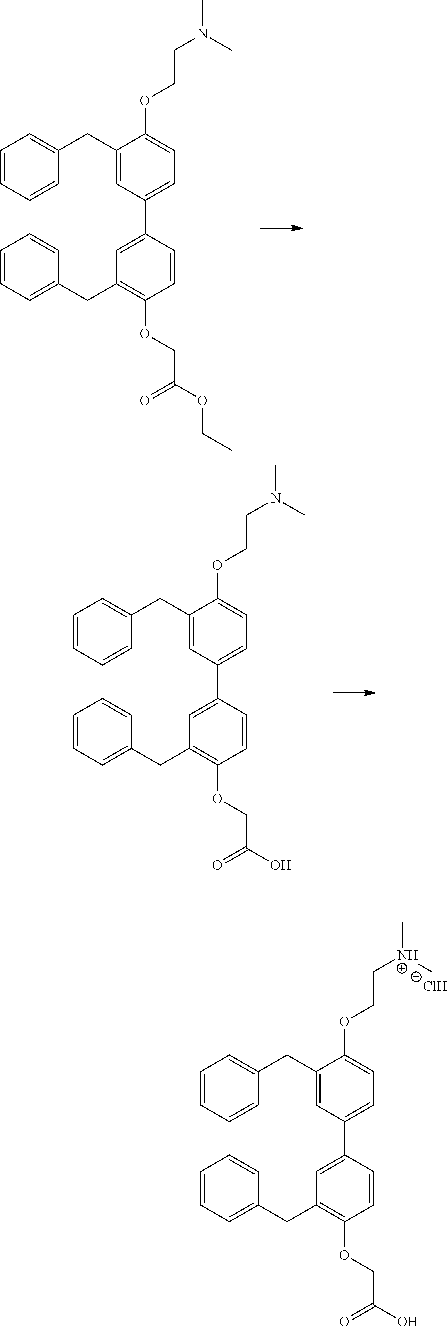

To a solution of ethyl 2-(1,1′-biphenyl-3,3′-dibenzyl-4′-[2-(dimethylamino)ethoxy]-4-oxy)acetate (7e) (25 mg, 0.048 mmol) in methanol or ethanol (3 mL) was added 1 N aqueous NaOH (0.10 mL, 0.10 mmol). The solution was heated to 40° C. for 2-4 h. The reaction was monitored by thin layer chromatography using C18 plates. Upon completion, the solution was concentrated under reduced pressure. The residue was acidified with 1 N HCl acid (25 mL) and extracted with ethyl acetate. The organic layer was then washed with water and brine, dried with magnesium sulfate, filtered and concentrated under reduced pressure yielding the carboxylic acid (21 mg, 88%).

The carboxylic acid of ethyl 2-(1,1′-biphenyl-3,3′-dibenzyl-4′-[2-(dimethylamino)ethoxy]-4-oxy)acetic acid (1e) (21 mg, 0.042 mmol) was dissolved in ethyl acetate or dioxane (2 ml). To this solution was added 4 N HCl acid in dioxane or ethyl acetate (3 ml). The reaction was allowed to stir at ambient temperature for 12-24 h. The solvent was removed under reduced pressure. The resulting solid was rinsed with minimal ethyl acetate and hexanes and the hydrochloride salt was collected by filtration (9 mg, 40%).

The compound was confirmed as follows: ¹H NMR (500 MHz, CD₃OD): δ=7.27 (s, 1H), 7.25 (s, 1H), 7.22-7.11 (m, 10H), 7.06 (s, 1H), 7.05 (s, 1H), 6.78 (d, J=8.5 Hz, 1H), 6.74 (d, J=9.0 Hz, 1H), 4.50 (s, 2H), 4.10 (t, J=5.3 Hz, 2H), 4.04 (s, 2H), 3.94 (s, 2H), 3.34 (t, 2H), 2.72 (s, 6H) ppm.

### 1. ethyl 2-(1,1′-biphenyl-3,3′-diisopropyl-4′-[2-(dimethylamino)ethoxy]-4-oxy)acetic acid (1b)

The procedure for 1e was repeated. The hydrolysis resulted in 100% conversion.

The compound was confirmed as follows: ¹H NMR (500 MHz, CD₃OD): δ=7.37 (d, J=1.5 Hz, 1H), 7.36 (d, J=2 Hz, 1H), 7.22 (dd, J=8.5, 2.5 Hz, 1H), 7.17 (dd, J=8.5, 2.5 Hz, 1H). 6.88 (d, J=8.5 Hz, 1H), 6.82 (d, J=8.0 Hz, 1H), 4.57 (s, 2H), 4.33 (t, J=4.5 Hz, 2H), 3.61 (t, J=4.5 Hz, 2H), 3.49 (7let, J=6.8 Hz, 1H), 3.39 (7let, J=6.8 Hz, 1H), 3.01 (s, 6H), 1.27 (d, J=7.0 Hz, 6H), 1.24 (d, J=7.5 Hz, 6H) ppm. Further conversion to the hydrochloride salt proceeded in 59% yield.

### 2. ethyl 2-(1,1′-biphenyl-3,3′-disec-butyl-4′-[2-(dimethylamino)ethoxy]-4-oxy)acetic acid (1c)

The procedure for 1e was repeated. The hydrolysis resulted in 72% yield.

The compound was confirmed as follows: ¹H NMR (500 MHz, CDCl₃-d): δ 7.34 (m, 4H), 7.07 (d, J=8.5 Hz, 1H), 6.90 (d, J=8 Hz, 1H), 4.70 (s, 2H), 4.41 (br s, 2H), 3.68 (br s, 2H), 3.24 (m, 2H), 3.06 (s, 6H), 1.70 (m, 4H), 1.28 (d, J=4.5 Hz, 2H), 1.27 (d, J=4 Hz, 2H), 0.90 (t. J=7.5 Hz, 3H), 0.89 (t, J=7.5 Hz, 3H) ppm. Further conversion to the hydrochloride salt proceeded in 40.1% yield.

### 3. 2-(1,1′-biphenyl-3,3′-ditert-butyl-4′-[2-(dimethylamino)ethoxy]-4-oxy)acetic acid (1d)

The procedure for 1e was repeated. The hydrolysis resulted in 88% yield.

The compound was confirmed as follows: ¹H NMR (500 MHz, CDCl₃-d): δ 7.37 (d, J=1.5 Hz, 1H), 7.34 (d, J=2.5 Hz, 1H), 7.26 (dd, J=8.5 Hz, 2.5 Hz, 1H), 7.20 (dd, J=8.5 Hz, 2 Hz, 1H), 6.98 (d, J=8 Hz, 1H), 6.79 (d, J=8.5 Hz, 1H), 4.57 (s, 2H), 4.33 (br t, J=4.5 Hz, 2H), 3.58 (br t, J=5 Hz, 2H), 2.95 (s, 6H), 1.36 (s, 9H), 1.35 (s, 9H) ppm. Further conversion to the hydrochloride salt proceeded in 78.8% yield.

### 4. Preparation of ethyl 2-(4-iodophenoxy)acetate (5a)

4-iodophenol (1.1 g, 5 mmol) was dissolved in dry THF (45 ml). Sodium hydride, 60% in oil, (0.28 g, 7 mmol) was added and the solution was stirred at RT for 30 minutes. 4-bromoethyl acetate (1.2 g, 7 mmol) was then added slowly and the solution was stirred for 1.5 h at RT. The reaction mixture (thick white solid) was diluted with ethyl acetate and washed with water (2×). After drying over magnesium sulfate, the product was chromatographed over silica gel (Hexane/EtOAc, 80:20) to afford 1.02 g (67%) of the desired product as an oil.

### 5. Preparation of ethyl 2-(1,1′-biphenyl-4′-ol-4-oxy)acetate 6a

2-(4-Iodo-phenyl-)-4-oxy)acetic acid (0.100 g, 1 equiv.), 4-hydroxyphenylboronic acid (0.0541 g, 1.2 equiv), PdCl₂(PPh₃)₂ (0.0230 g, 10 mol %), triphenyl phosphine (0.00858 g, 10 mol %) and 2M sodium carbonate (0.0858 g and 4-hydroxyphenylboronic acid (0.0541 g, 1.2 equiv.) were combined in a sealed vial and dissolved in 4 mL THF and 1 mL H₂O. The reaction underwent microwave irradiation at 110° C. for 20 minutes using a Biotage Initatiator™ 2.0. The crude reaction mixture was concentrated and purified by silica gel chromatography (10% EtOAc/Hex) to afford 0.030 g (34%) of the desired product as a white solid.

### 6. ethyl 2-(1,1′-biphenyl-4′-[2-(dimethylamino)ethoxy]-4-oxy)acetate (7a)

The procedure for 7e was repeated. Product was recovered in 73.7% yield as a white solid.

### 7. ethyl 2-(1,1′-biphenyl-4′-[2-(dimethylamino)ethoxy]-4-oxy)acetic acid (1a)

8a was isolated as a sodium salt by stopping the procedure of 8e after the initial hydrolysis step and recovering the salt from ethyl acetate.

### EQUIVALENTS

Those skilled in the art will recognize, or be able to ascertain, using no more than routine experimentation, numerous equivalents to the specific embodiments described specifically in this disclosure. Such equivalents are intended to be encompassed in the scope of the following claims.

1. A compound of Formula (I), 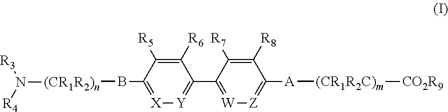
and pharmaceutically acceptable salts, hydrates, solvates, tautomers, and prodrugs thereof,
wherein:
A and B are each independently a bond or —O—;
R₁ and R₂ are each independently H or alkyl;
R₃ and R₄ are each independently H, alkyl, cycloalkyl, aralkyl, —C(O)alkyl, —C(O)NH(alkyl), —C(O)N(alkyl)₂;
R₅, R₆, R₇, and R₈ are each independently H, alkyl, aralkyl, or cycloalkyl;
n and m are each independently an integer from 1-6;
W, X, Y, and Z are each independently CH, N, or CO₂H; and
R₉ is H, alkyl, aralkyl, —NH-alkyl, —N(alkyl)₂, —NH-aminoacid, or azole.

2. The compound of claim 1, having the structure of Formula (IA), 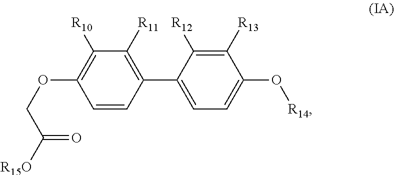
and pharmaceutically acceptable salts, hydrates, solvates, tautomers, and prodrugs thereof,
wherein:
R₁₀, R₁₁, R₁₂, and R₁₃ are each independently H, C₁-C₄alkyl, or C₁-C₄alkyl-aryl;
R₁₄ is H or C₁-C₄alkyl-dialkyl(C₁-C₄)amino; and
R₁₅ is H or C₁-C₄alkyl.

3. The compound of claim 2, wherein R₁₀ is isopropyl, sec-butyl, or tert-butyl.

4. The compound of claim 2, wherein R₁₁ is isopropyl, sec-butyl, or tert-butyl.

5. The compound of claim 2, wherein R₁₀ is benzyl and R₁₁ is benzyl.

6. (canceled)

7. The compound of claim 2, wherein R₁₂ is isopropyl, sec-butyl, or tert-butyl.

8. The compound of claim 2, wherein R₁₃ is isopropyl, sec-butyl, or tert-butyl.

9. The compound of claim 2, wherein R₁₂ is benzyl and R₁₃ is benzyl.

10. (canceled)

11. The compound of claim 2, wherein R₁₄ is

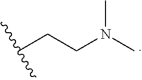

12. The compound of claim 2, wherein R₁₅ is ethyl.

13. A method of disrupting androgen or estrogen signaling, comprising:
(a) obtaining a sample containing an androgen or estrogen receptor and a co-regulatory protein;
(b) contacting the sample with the compound of Formula (I) of claim 1;
(c) detecting an amount of co-regulatory protein-androgen or protein-estrogen receptor complex formed; and
(d) comparing the amount of complex formed in (c) to an amount of complex formed in a control sample that does not contain the compound of Formula (I),
wherein androgen or estrogen signaling is disrupted when the amount of complex in the sample is detectably less than the amount of complex formed in the control sample.

14. The method of claim 13, wherein the compound Formula (I) having the structure of Formula (IA), 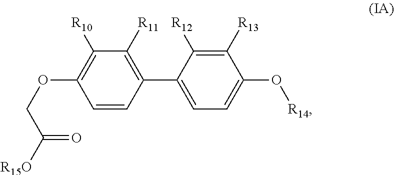
or a pharmaceutically acceptable salt, hydrate, solvate, tautomer, or prodrug thereof, wherein:
R₁₀, R₁₁, R₁₂, and R₁₃ are each independently H, C₁-C₄alkyl, or C₁-C₄alkyl-aryl;
R₁₄ is H or C₁-C₄alkyl-dialkyl(C₁-C₄)amino; and
R₁₅ is H or C₁-C₄alkyl.

15-36. (canceled)

37. A method of inhibiting the interaction between a ligand-bound andogen or estrogen receptor and a co-regulatory protein, comprising:
(a) obtaining a sample containing a ligand-bound androgen or estrogen receptor and a co-regulatory protein;
(b) contacting the sample with a the compound of Formula (I) of claim 1;
(c) detecting an amount of co-regulatory protein-androgen or protein-estrogen receptor complex formed; and
(d) comparing the amount of complex formed in (c) to an amount of complex formed in a control sample that does not contain the compound of Formula (I),
wherein the interaction between a ligand-bound andogen or estrogen receptor or and a co-regulatory protein is inhibited when the amount of complex in the sample is detectably less than the amount of complex formed in the control sample.

38. The method of claim 37, wherein the compound of Formula (I) having the structure of Formula (IA), 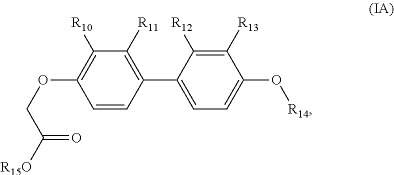
or a pharmaceutically acceptable salt, hydrate, solvate, tautomer, or prodrug thereof, wherein:
R₁₀, R₁₁, R₁₂, and R₁₃ are each independently H, C₁-C₄alkyl, or C₁-C₄alkyl-aryl;
R₁₄ is H or C₁-C₄alkyl-dialkyl(C₁-C₄)amino; and
R₁₅ is H or C₁-C₄alkyl.

39-60. (canceled)

61. A method of inhibiting cell proliferation in cancer cells, comprising:
(a) contacting a sample containing a cancer cell with the compound of Formula (I) of claim 1; and
(b) detecting the proliferative state of the cell in the sample, cell stasis or death in the sample being indicative of the inhibition of cell proliferation.

62. The method of claim 61, wherein the compound of Formula (I) having the structure of Formula (IA), 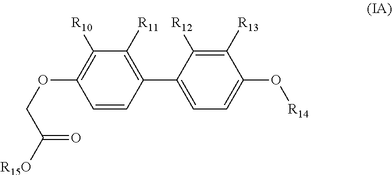
or a pharmaceutically acceptable salt, hydrate, solvate, tautomer, or prodrug thereof, wherein:
R₁₀, R₁₁, R₁₂, and R₁₃ are each independently H, C₁-C₄alkyl, or C₁-C₄alkyl-aryl;
R₁₄ is H or C₁-C₄alkyl-dialkyl(C₁-C₄)amino; and
R₁₅ is H or C₁-C₄alkyl.

63. A method of treating cancer in a subject, comprising:
(a) administering to the subject a therapeutically effective amount of the compound of Formula (I) of claim 1; and
(b) detecting a decrease in a symptom of the cancer, the compound treating the cancer by inhibiting androgen or estrogen signaling in the cancer cell.

64. The method of claim 63, wherein the cancer is prostate cancer.

65. The method of claim 63, wherein the cancer is breast cancer.

66. The method of claim 63, wherein the compound of Formula (I) having the structure of Formula (IA): 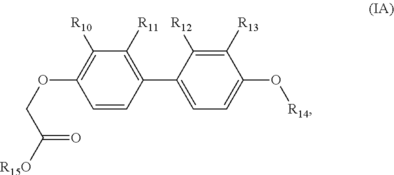
or a pharmaceutically acceptable salt, hydrate, solvate, tautomer, or prodrug thereof, wherein:
R₁₀, R₁₁, R₁₂, and R₁₃ are each independently H, C₁-C₄alkyl, or C₁-C₄alkyl-aryl;
R₁₄ is H or C₁-C₄alkyl-dialkyl(C₁-C₄)amino; and
R₁₅ is H or C₁-C₄alkyl.

67-74. (canceled)
